(19) United States
(12) Patent Application Publication
(10) Pub. No.: US 2025/0312296 A1
(43) Pub. Date: Oct. 9, 2025

(54) PETROLATUM-BASED PHMB COMPOSITIONS AND METHODS FOR THE TREATMENT OF INFLAMMATORY SKIN DISEASE

(71) Applicant: GLOBAL HEALTH SOLUTIONS, INC., Westlake Village, CA (US)

(72) Inventor: Bradley Burnam, Calabasas, CA (US)

(21) Appl. No.: 19/243,547

(22) Filed: Jun. 19, 2025

Related U.S. Application Data
(63) Continuation-in-part of application No. 17/468,809, filed on Sep. 8, 2021.
Application No. 17/468,809 is a continuation of application No. 16/745,308, filed on Jan. 16, 2020, now Pat. No. 11,116,734.
(60) Provisional application No. 63/662,319, filed on Jun. 20, 2024.

(51) Int. Cl. A61K 31/14; A61K 31/135; A61K 31/4164; A61K 31/4412; A61K 45/06; A61K 47/06; A61P 31/10

(52) U.S. Cl. CPC A61K 31/14; A61K 31/135; A61K 31/4164; A61K 31/4412; A61K 45/06; A61K 47/06; A61P 31/10

(57) ABSTRACT

Compositions and methods for the treatment of inflammatory skin diseases. The compositions include a pharmaceutically effective amount of polyhexamethylene biguanide (PHMB), polyaminopropyl biguanide (PAPB), and/or chlorhexidine (CHG) in a petrolatum carrier.

### CROSS-REFERENCE TO RELATED APPLICATIONS

This application is a continuation-in-part of U.S. patent application Ser. No. 17/468,809, filed Sep. 8, 2021, which is a continuation of U.S. patent application Ser. No. 16/745,308, now U.S. Pat. No. 11,116,734, filed Jan. 16, 2020, which is a continuation-in-part of U.S. patent application Ser. No. 15/167,099, now U.S. Pat. No. 10,722,461, filed May 27, 2016, which claims priority to U.S. Provisional Application No. 62/182,034, filed Jun. 19, 2015, U.S. Provisional Application No. 62/319,449, filed Apr. 7, 2016, U.S. Provisional Application No. 62/326,150, filed Apr. 22, 2016, and U.S. Provisional Application No. 62/338,995, filed May 19, 2016. This application also claims the benefit of U.S. Provisional Application Ser. No. 63/662,319, entitled “Petrolatum-Based Compositions and Methods for the Treatment of Inflammatory Skin Disease,” filed Jun. 20, 2024. The contents of each of the aforementioned patent applications are incorporated by reference herein, for all purposes, in their entirety.

### FIELD

The present disclosure is broadly concerned with petrolatum-based cationic-biocide compositions for the treatment and prevention of inflammatory skin disease. The disclosure is also concerned with methods for the treatment of inflammatory skin disease using petrolatum-based polihexanide biguanide (PHMB) compositions.

### BACKGROUND

Inflammatory skin diseases represent the largest class of chronic skin disease and can be expensive to treat and be characterized by a poor prognosis. Common inflammatory skin diseases include eczema, psoriasis, rosacea, lichen planus, seborrheic dermatitis, acene vulgaris, urticaria, hidradenitis suppurativa, folliculitis, dermatitis herpetiformis, alopecia areata, and vitiligo. Atopic dermatitis, often referred to as eczema, is one of the most common inflammatory skin diseases. Eczema is a chronic disease that causes inflammation, redness, and irritation of the skin and can affect people at any age.

Inflammatory skin diseases are often characterized by the activation of the innate and adaptive immune system via the production of pro-inflammatory cytokines, such as interleukin (IL)-36 cytokines, IL-31, and IL-4. In particular, it has been shown that pro-inflammatory cytokines are key initiators of immune responses and pathological inflammation within epithelial tissues. For example, IL-31 and IL-36 alpha are pro-inflammatory cytokines that are known to play a significant role in skin inflammation and eczema (atopic dermatitis). Similarly, IL-36 gamma has been shown to play a significant role in the development and progression of psoriasis and is highly expressed in active psoriatic lesions and can drive inflammatory processes in skin models. Pro-inflammatory cytokines are also generally known to play a role in the development of red, irritated, and itchy skin as well.

Conventional treatments for atopic dermatitis and other inflammatory skin diseases treat the symptoms and secondary inflammatory pathways but do not intervene in the source of the inflammation and disease pathogenesis. Accordingly, additional compositions and methods for the treatment of inflammatory skin diseases are desirable. Non-steroid and non-injectable treatment options that are effective at the site of disease in the skin are particularly desirable. Additionally, topical treatment compositions operable to prevent or treat skin inflammation in a subject by mitigating or reducing the expression of one or more pro-inflammatory cytokines are desirable.

### BRIEF DESCRIPTION OF THE FIGURES

FIG. 1 depicts a box plot statistical analysis of IGA score data of Group 2 (negative control) versus Group 3 (treatment with presently disclosed PHMB Petrolatum-Based Composition) with each group mean values and p-values indicated, according to an exemplary embodiment of the present disclosure.

FIG. 2 depicts a Western Blot protein expression analysis for IL36-alpha protein expression in Group 1 (negative control) and Group 2 (pre-treatment with Formulation 1), according to an exemplary embodiment of the present disclosure.

FIG. 3 depicts a Western Blot protein expression analysis for IL36-gamma protein expression in Group 1 (negative control) and Group 2 (pre-treatment with Formulation 1), according to an exemplary embodiment of the present disclosure.

FIG. 4 depicts a Western Blot protein expression analysis for IL31 protein expression in Group 1 (negative control) and Group 2 (pre-treatment with Formulation 1), according to an exemplary embodiment of the present disclosure.

FIG. 5 depicts a Western Blot protein expression analysis for IL4 protein expression in Group 1 (negative control) and Group 2 (pre-treatment with Formulation 1), according to an exemplary embodiment of the present disclosure.

FIG. 6 depicts a Western Blot protein expression analysis for IL13 protein expression in Group 1 (negative control) and Group 2 (pre-treatment with Formulation 1), according to an exemplary embodiment of the present disclosure.

FIG. 7 depicts a Raw Integrated Density plot for IL36-alpha protein expression in Group 1 (negative control) and Group 2 (pre-treatment with Formulation 1), according to an exemplary embodiment of the present disclosure.

FIG. 8 depicts a Raw Integrated Density plot for IL36-gamma protein expression in Group 1 (negative control) and Group 2 (pre-treatment with Formulation 1), according to an exemplary embodiment of the present disclosure.

FIG. 9 depicts a Raw Integrated Density plot for IL31 protein expression in Group 1 (negative control) and Group 2 (pre-treatment with Formulation 1), according to an exemplary embodiment of the present disclosure.

FIG. 10 depicts a Raw Integrated Density plot for IL4 protein expression in Group 1 (negative control) and Group 2 (pre-treatment with Formulation 1), according to an exemplary embodiment of the present disclosure.

FIG. 11 depicts a Raw Integrated Density plot for IL13 protein expression in Group 1 (negative control) and Group 2 (pre-treatment with Formulation 1), according to an exemplary embodiment of the present disclosure.

FIG. 12 depicts a box plot summary of a IGA score comparison for Group 1 (negative control) and Group 2 (pre-treatment with Formulation 1) with each group mean values and standard deviation values indicated, according to an exemplary embodiment of the present disclosure.

### DETAILED DESCRIPTION

The present disclosure provides compositions and methods for the treatment of inflammatory skin diseases. A non-limiting list of inflammatory skin diseases that may be treated with the presently disclosed compositions and methods may include, for example, eczema, psoriasis, rosacea, lichen planus, seborrheic dermatitis, acne vulgaris, urticaria, hidradenitis suppurativa, folliculitis, dermatitis herpetiformis, alopecia areata, and vitiligo. In some instances, the inflammatory skin disease is eczema. In such cases, the present disclosure provides methods and compositions for the treatment of eczema. The presently disclosed methods include applying a petrolatum-based polyhexamethylene biguanide (PHMB) composition to the skin of a subject in need of treatment. The presently disclosed petrolatum-based PHMB compositions may be applied to the skin of a subject, or portion thereof, in need of treatment in any manner reasonably expected to result in topical contact of the petrolatum-based PHMB compositions with the skin of a subject, or portion thereof, in need of treatment.

The present disclosure further provides methods and compositions for controlling, mitigating, or reducing inflammation in the skin of a subject, as well as methods and compositions for controlling, mitigating, or reducing the production of one or more pro-inflammatory cytokines in a subject. The presently disclosed methods include applying a petrolatum-based polyhexamethylene biguanide (PHMB) composition to the skin of a subject in need of treatment. In certain embodiments, the one or more pro-inflammatory cytokines may be selected from the group consisting of IL-36 alpha, IL-36 gamma, IL-31, and IL-4.

In some embodiments, the methods and compositions may be applied to the skin of a subject, or a portion thereof, that is expected, known, or determined to be colonized by a yeast, fungus, bacteria, or other microorganism. Examples of yeast, fungus, bacteria, or other microorganism may include Staphylococcus aureus (S. aureus), Staphylococcus epidermis (S. epidermis), micrococci, diphtheroids (coryneforms), streptococci, gram-negative bacilli (such as Enterobacter, Klebsiella, Escherichia coli, Proteus spp., and Acinetobacter spp), Aspergillus, Penicillium, Cladosporium, Mucor, Propionibacterium acnes, P. granulosum, P. avidum, Corynebacterium spp., and Micrococcus luteus. In certain embodiments, the yeast, fungus, bacteria, or other microorganism is Staphylococcus aureus (S. aureus). In certain specific embodiments, the inflammation, inflammatory skin disease, or pro-inflammatory cytokines production may be associated with or expected to be caused by Staphylococcus aureus (S. aureus).

It has been unexpectedly discovered that the presently disclosed compositions comprising polyhexamethylene biguanide (PHMB) are effective in the treatment of inflammatory skin diseases and/or for controlling, mitigating, or reducing inflammation in the skin of a subject. In particular, it has unexpectedly been discovered that the presently disclosed topical compositions and methods are effective in preventing or treating inflammation and inflammatory skin disease in the subject by controlling, mitigating, or reducing the production of one or more pro-inflammatory cytokines in the skin of the subject, thereby directly intervening at the site of disease pathogenesis. The presently disclosed compositions and methods provide a non-steroid, non-injectable treatment option that intervenes at the disease source by reducing the production or expression of pro-inflammatory cytokines such as IL-36 alpha, IL-36 gamma, IL-31, and IL-4. In particular, the presently disclosed compositions and methods are effective for the treatment of atopic dermatitis by reducing or mitigating the expression of IL-31 and IL-36 alpha pro-inflammatory cytokines in the skin of the subject. Similarly, the presently disclosed compositions and methods are effective for the treatment of psoriasis by reducing or mitigating the expression of IL-36 gamma pro-inflammatory cytokines in the skin of the subject.

The petrolatum-based PHMB composition applied to the skin of the subject according to the presently disclosed methods, may include, for example, greater than about 80% by weight petrolatum, or greater than about 85% by weight petrolatum, or greater than about 90% by weight petrolatum, or greater than about 95% by weight petrolatum. The petrolatum-based PHMB composition may also include from about 0.005% to about 5% by weight PHMB, or from about 0.01% to about 5% by weight PHMB, or from about 0.05% to about 5% by weight PHMB, or from about 0.05% to about 3% by weight PHMB, or from about 0.1% to about 1% by weight PHMB, or from about 0.2% to about 0.6% by weight PHMB, or from about 0.3% to about 0.5% by weight PHMB, or from about 0.1% to about 3.5% by weight PHMB, or from about 0.05% to about 2.5% by weight PHMB, or from about 0.5% to about 3% by weight PHMB, or from about 0.5% to about 2.5% by weight PHMB, or from about 1.5% to about 2.5% by weight PHMB. In at least some instances, the petrolatum-based PHMB composition contains no emulsifier. In other instances, the petrolatum-based PHMB composition excludes an added emulsifier. As used herein, the term “added emulsifier” refers to an emulsifier in addition to the presently claimed components of the petrolatum-based PHMB composition.

PHMB is closely related to the polymeric biguanide polyaminopropyl biguanide (PAPB). Therefore, in at least some instances, a pharmaceutically effective amount of polyaminopropyl biguanide (PAPB) may be substituted for the pharmaceutically effective amount of PHMB in the presently disclosed compositions and methods. For example, the petrolatum-based PHMB composition may include from about 0.005% to about 5% by weight PAPB, or from about 0.01% to about 5% by weight PAPB, or from about 0.05% to about 5% by weight PAPB, or from about 0.05% to about 3% by weight PAPB, or from about 0.1% to about 1% by weight PAPB, or from about 0.2% to about 0.6% by weight PAPB, or from about 0.3% to about 0.5% by weight PAPB, or from about 0.1% to about 3.5% by weight PAPB, or from about 0.05% to about 2.5% by weight PAPB, or from about 0.5% to about 3% by weight PAPB, or from about 0.5% to about 2.5% by weight PAPB, or from about 1.5% to about 2.5% by weight PAPB.

The petrolatum-based PHMB composition may also include from about from about 0.05% to about 5% by weight PHMB, or from about 0.05% to about 3% by weight PHMB, or from about 0.1% to about 1% by weight PHMB, or from about 0.2% to about 0.6% by weight PHMB, or from about 0.3% to about 0.5% by weight PHMB, or from about 0.1% to about 3.5% by weight PHMB, or from about 0.05% to about 2.5% by weight PHMB, or from about 0.5% to about 3% by weight PHMB, or from about 0.5% to about 2.5% by weight PHMB, or from about 1.5% to about 2.5% by weight PHMB.

In at least some instances, chlorhexidine may be substituted for some portion of the PHMB or PAPB present in the petrolatum-based PHMB composition. In at least some instances, the petrolatum-based PHMB composition may further include a cationic biocide selected from the group consisting of benzalkonium chloride, cetrimide, chlorhexidine, and any combination thereof. In at least some instances, the petrolatum-based PHMB composition may further include a preservative selected from the group consisting of benzalkonium chloride, cetrimide, chlorhexidine, and any combination thereof. In some instances, the petrolatum-based PHMB composition may include from about 0.001% to about 0.01% by weight benzalkonium chloride (BZK), or from about 0.001% to about 0.15% by weight BZK, or from about 0.005% to about 0.007% by weight BZK. In at least some instances, the BZK is included as a preservative in the petrolatum-based PHMB composition. In at least some instances, the petrolatum-based PHMB composition may be prepared by a process that includes: (a) dissolving the PHMB in a polar solvent to give a PHMB solution; (b) heating the petrolatum to a temperature sufficient to cause the petrolatum to melt to give a melted petrolatum and heating the PHMB solution to a temperature higher than the temperature of the melted petrolatum to give a heated PHMB solution; (c) mixing the melted petrolatum and the heated PHMB solution to give a melted mixture; and (d) cooling the melted mixture to give the petrolatum-based PHMB composition. In some instances, the heated PHMB solution may have a temperature that is about 1° C. to about 5° C. higher than the temperature of the melted petrolatum. In some instances, the PHMB is dissolved in a polar solvent to form a PHMB solution and the PHMB solution is dispersed in the petrolatum to form a stable suspension. The polar solvent may be, for example, water, ethanol, or any combination of water and ethanol.

According to at least one aspect of the present disclosure, the petrolatum-based PHMB compositions may further include one or more additional anti-fungal agents selected from the group consisting of terbinafine HCl, ciclopirox, ciclopirox olamine, fluconazole, itraconazole, ketoconazole, amorolfine, efinaconazole, clotrimazole, miconazole (miconazole nitrate), and any combination thereof. In some instances, the petrolatum-based PHMB composition may include just one additional anti-fungal agent. In such instances, the petrolatum-based PHMB composition may include from about 0.5% to about 5% by weight, or from about 0.5% to about 1.5% by weight, or from about 0.75% to about 1.25% by weight, or from about 0.5% to about 2.5% by weight additional anti-fungal agent based on the total weight of the petrolatum-based PHMB composition. In other instances, the petrolatum-based PHMB composition may include more than one additional anti-fungal agents. In such instances, the total weight of the additional anti-fungal agents in the petrolatum-based PHMB composition may be from about 0.5% to about 5% by weight, or from about 0.5% to about 1.5% by weight, or from about 0.75% to about 1.25% by weight, or from about 0.5% to about 2.5% by weight based on the total weight of the petrolatum-based PHMB composition.

According to at least one aspect of the present disclosure, the petrolatum-based PHMB composition may be a petrolatum-based composition that includes petrolatum and a pharmaceutically effective amount of PHMB. In such cases, the compositions may include, for example, greater than about 80% by weight petrolatum. The compositions may also include a polar solvent. The polar solvent may comprise from about 1% to about 15% by weight, or from about 1% to about 5% by weight, or from about 1% to about 10% by weight, or from about 1% to about 7% by weight, or from about 2.5% to about 5% by weight, or from about 2% to about 6% by weight of the petrolatum-based PHMB composition. In some instances, the polar solvent may be water. In other instances, the polar solvent may be ethanol. In still other instances, the polar solvent may be a mixture of water and ethanol, in any proportion, but preferably in a water to ethanol ratio of from about 60:40 to about 90:10 by weight. In at least some instances, ethanol may comprise from about 10% to about 40% by weight of the PHMB solution. When used as polar solvent, ethanol may comprise from about 0.01% to about 6.5% by weight, or from about 0.5% to about 2.5% by weight, or from about 0.5% to about 1.5% by weight of the petrolatum-based PHMB composition.

In certain cases, the PHMB, and the polar solvent may be dispersed in the petrolatum in the form of nanodroplets. According to at least one aspect, the petrolatum-based PHMB compositions contain no emulsifier. In some instances, the PHMB, may be dissolved in a polar solvent to form a PHMB solution and the PHMB solution dispersed in the petrolatum. In such cases, the PHMB solution may be dispersed in the petrolatum to form a stable suspension such that the PHMB solution does not separate from the petrolatum for at least six months. According to at least one aspect of the present disclosure, the resultant petrolatum-based PHMB composition does not require an emulsifier to form a stable suspension of PHMB dispersed in the petrolatum. Further, the petrolatum-based PHMB composition prepared according to this process does not require high shear mixing to form a stable suspension of PHMB in petrolatum in the absence of an added emulsifier.

### I. Compositions

According to one aspect, the present disclosure provides for compositions that are petrolatum-based. A petrolatum-based composition is made up primarily of petrolatum. The characteristics of a petrolatum-based composition differ from a composition containing only a small amount of petrolatum. In some embodiments, the petrolatum-based composition is greater than about 80% petrolatum. In other embodiments, the petrolatum-based composition is greater than about 81% petrolatum, greater than about 82% petrolatum, greater than about 83% petrolatum, greater than about 84% petrolatum, greater than about 85% petrolatum, greater than about 86% petrolatum, greater than about 87% petrolatum, greater than about 88% petrolatum, greater than about 89% petrolatum, greater than about 90% petrolatum, greater than about 91% petrolatum, greater than about 92% petrolatum, greater than about 93% petrolatum, greater than about 94% petrolatum, greater than about 95% petrolatum, greater than about 96% petrolatum, greater than about 97% petrolatum, greater than about 98% petrolatum, or greater than about 99% petrolatum. The petrolatum is preferably medical grade petrolatum.

The composition also contains PHMB dispersed throughout the petrolatum. PHMB is the composition ingredient active in treating inflammatory skin disease. In addition to PHMB, the compositions may also include other cationic biocides, such as quaternary ammonium compounds, bisbiguanides, and polymeric biguanides. In particular, other cationic biocides that may be included in the compositions may include, but are not limited to, benzalkonium chloride, cetrimide, chlorhexidine, polihexanide biguanide (polihexanide, polyhexamethylene guanide, poly(iminoimidocarbonyl-iminoimidocarbonyl-iminohexamethylene), poly(hexamethylenebiguanide), polyaminopropyl biguanide) and salts or combinations thereof. In one embodiment, the composition contains a mixture of polihexanide biguanide (PMHB) and benzalkonium chloride (BZK) preservative. The total amount of cationic biocide in the composition generally constitutes less than about 1% by weight of the total composition. In preferred embodiments, the cationic biocide constitutes from about 0.1% to about 0.5% by weight, or more preferably, from about 0.1% to about 0.3% by weight to the total composition.

The remaining weight of the composition, typically from about 0.1% to about 6% by weight of the petrolatum-based composition, is liquid. In one embodiment, the composition contains about 5% water.

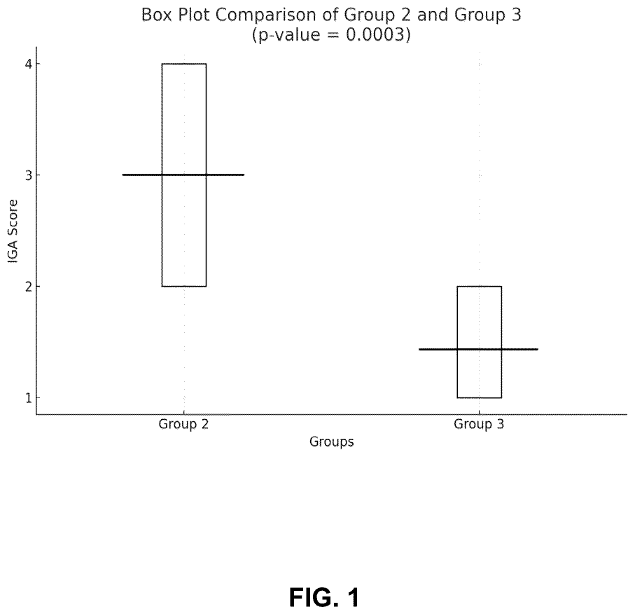

The PHMB and other cationic biocides that may be used do not react with the petrolatum. Instead, the PHMB and other cationic biocides and/or other antifungal agents that may be included in the compositions are dispersed in the petrolatum as nanodroplets, and the petrolatum serves as a suspension matrix for the PHMB and other cationic biocides. “Nanodroplet,” as used herein, is an aggregation of PHMB and optionally any cationic biocide molecules or antifungal agents in the petrolatum base. The nanodroplets typically contain a small amount of water in addition to the PHMB and other optional cationic biocides or additional antifungal agents. Nanodroplets in accordance with the present disclosure are shown in FIG. 1. The nanodroplets may vary in size but generally the longest dimension of the nanodroplets measures from about 10 nm to about 10,000 nm. In various embodiments, the nanodroplets range from about 10 nm to about 100 nm, from about 100 nm to about 1000 nm, from about 1000 nm to about 2000 nm, from about 2000 nm to about 3000 nm, from about 3000 nm to about 4000 nm, from about 4000 nm to about 5000 nm, from about 5000 nm to about 6000 nm, from about 6000 nm to about 7000 nm, from about 7000 nm to about 8000 nm, from about 8000 nm to about 9000 nm, from about 9000 nm to about 10,000 nm. The nanodroplets are dispersed through the petrolatum homogeneously.

Surprisingly, embodiments of the present invention do not require an emulsifier. An emulsifier, as used herein, is an added formulation ingredient used to reduce the tension between hydrophilic and hydrophobic surface ingredients, thereby facilitating the mixture hydrophilic and hydrophobic ingredients. Prior to the present invention, those skilled in the art expected that an emulsifier would be needed to disperse cationic biocides, such as PHMB, which are polar, as well as additional anti-fungal agents, in a non-polar petrolatum suspension matrix. Where an emulsifier is used, the emulsifier may have a hydrophilic-lipophilic balance (HLB) of less than 10.

The compositions described herein are stable. In one aspect, stability refers to the integrity of the composition as a whole, and in particular, the stability of the nanodroplets in the petrolatum. Under ambient conditions, the petrolatum and the cationic biocides will not separate for greater than two years, meaning that the composition is shelf stable for at least two years. Even under accelerated conditions, such as reduced pressure, the petrolatum and the PHMB and any other cationic biocides do not separate, but rather the PHMB and cationic biocides, and optional additional anti-fungal agents, remain suspended as nanodroplets in the petrolatum. In addition to the stability of the nanodroplets within the composition, the compositions described herein also show exceptional chemical stability for the PHMB and other cationic biocides. The chemical stability stems primarily from the low-temperature manufacturing process described below. The absence of excessive heat conditions in the manufacturing of the compositions improves the chemical stability (resistance to degradation) for the PHMB and other cationic biocides.

In some embodiments, the petrolatum-based compositions described herein consist essentially of petrolatum, PHMB, and water. In one preferred embodiment, the petrolatum-based compositions consist essentially of petrolatum, benzalkonium chloride, polihexanide biguanide, and water. In alternative embodiments, the petrolatum-based compositions described herein consist of petrolatum, a cationic biocide, and water or consist of petrolatum, benzalkonium chloride, polihexanide biguanide, and water.

In other embodiments, the petrolatum-based compositions described herein may further comprise a compound that stimulates healing. More specifically, the petrolatum-based compositions described herein may further comprise a compound that stimulates healing for use in intraoperative applications and chronic wound care applications. Non-limiting examples of compounds that stimulate healing include polycaprolactone-tricalcium phosphate (PCL-TCP), collagen, chitosan, cellulose, thrombin, chondroitin sulfate (CS), chondroitin sulfate succinimidyl succinate (CS-NHS), and growth factors such as TGF-alpha, TGF-beta (TGFβ1, TGFβ2, TGFβ3), platelet-derived growth factor (PDGF), epidermal growth factor (EGF), fibroblast growth factor also referred to as keratinocyte growth factor (FGF1, FGF2, FGF4, FGF7), vascular endothelial growth factor (VEGF), insulin-like growth factor (IGF), connective tissue growth factor (CTGF), activin, interleukin-1 (IL1α, IL1β), TNFα, GM-CSF, or autologous intraoperative biologics such as platelet-rich plasma (PRP) and bone marrow (BM).

In other embodiments, the petrolatum-based compositions described herein may further comprise a dermatologically acceptable carrier. A “dermatologically-acceptable carrier,” as used herein, is a component or components suitable for use in contact with human keratinous tissue without undue toxicity, incompatibility, instability, allergic response, and the like. Where employed, the carrier is inert in the sense of not bringing about a deactivation of the active ingredients, and in the sense of not bringing about any adverse effect on the skin areas to which it is applied. Common dermatological additives are also envisioned for some embodiments. In certain embodiments, a dermatological additive is a whitening agent and/or hemostatic agent.

Additionally, the compositions may be incorporated in predetermined therapeutically effective amounts into disposables such as wipes, gauze, patches, wraps, bandages, adhesive strips, sponge, cotton swab, glove, sock, wrist bands, fabric, fibers, sutures, medication pad, underwear, tissue, pain-relief gel pack or bed liner and the like. For instance, the composition may be applied to the surface of, or impregnated into disposables.

### II. Process for Making

The disclosure also provides a method for making the compositions described in Section (I). The process comprises: (a) dissolving PHMB in a solvent to give a PHMB solution; (b) heating the petrolatum to a temperature sufficient to give a melted petrolatum, and heating the PHMB solution to a temperature higher than the temperature of the petrolatum to give a heated PHMB solution; (c) mixing the melted petrolatum and the heated PHMB solution to give a melted mixture; and, (d) cooling the melted mixture to give the petrolatum-based composition. As would be appreciated by one of skill in the art, steps (a)-(d) are conducted sequentially.

The PHMB as well any other cationic biocide, selected from the group described in Section (I), is first dissolved in a solvent to give a PHMB/cationic biocide solution. Acceptable solvents for the PHMB/cationic biocide solution include water or other solvents. Generally polar solvents such as water, ethanol, or any combination thereof are used. The PHMB and any other cationic biocides are typically dissolved in the solvent a concentration ranging from about 0.005% to about 5%. Typically, the amount of solvent used is from about 1:10 to about 1:30 the amount of petrolatum and more preferably is about 1:20 to the amount of petrolatum by volume. The amount of PHMB and other cationic biocides can be calculated by one skilled in the art to provide the desired weight percentage for the final composition.

Both the PHMB solution and the petrolatum are heated. The heating of these two ingredients can be conducted at the same time or sequentially so long as the melted petrolatum and the heated PHMB solution are at the appropriate temperatures during the mixing step. Petrolatum is a solid that melts at approximately 37° C. As such, petrolatum may be heated to any temperature at or above 37° C. For instance, the petrolatum may be heated to a temperature ranging from about 37° C. to about 45° C., from about 40° C. to about 50° C., from about 45° C. to about 55° C., from about 50° C. to about 60° C., from about 55° C. to about 65° C., from about 60° C. to about 70° C., from about 65° C. to about 75° C., from about 70° C. to about 80° C., from about 75° C. to about 85° C., from about 80° C. to about 90° C., from about 85° C. to about 95° C., or from about 90° C. to about 100° C. or more. Higher temperatures may also be envisioned. Preferably, the petrolatum is heated to a temperature ranging from about 37° C. to about 55° C., more preferably to a temperature ranging from about 40° C. to about 50° C. Heat may be provided to the petrolatum by any method known in the art, but a water bath or low temperature hot plate are preferred.

The PHMB solution is heated to a temperature above the temperature of the melted petrolatum. Any temperature above the temperature of the melted petrolatum may be used in a method of the present disclosure, provided that the heat does not cause excessive degradation of an active ingredient such as PHMB, or excessive evaporation of the active ingredient or polar solvent. For instance, the PHMB solution may be heated to a temperature that is about 1° C. to about 10° C., about 5° C. to about 15° C., about 10° C. to about 20° C., about 15° C. to about 25° C., about 20° C. to about 30° C., about 25° C. to about 35° C., about 30° C. to about 40° C., about 35° C. to about 45° C., about 40° C. to about 50° C., about 45° C. to about 55° C., about 50° C. to about 60° C. or about 65° C. or about 75° C. higher than the temperature of the melted petrolatum. Higher temperatures may also be envisioned. Preferably, the PHMB solution is heated to a temperature that is about 1° C. to about 10° C. higher than the temperature of the melted petrolatum. In another embodiment, the PHMB solution is heated to a temperature that is about 1° C. to about 5° C. higher than the melted petrolatum. In still other embodiments, the PHMB solution is heated to a temperature that is about 1° C., 2° C., 3° C., 4° C., or 5° C. above the temperature of the melted petrolatum. Again, the heating can be provided by any means known in the art but is preferably provided by a water bath or low temperature hot plate.

Once both the petrolatum and the PHMB solution are heated as described above, the melted petrolatum and the heated PHMB solution are mixed to give a melted mixture containing petrolatum and the heated PHMB solution. The mixing can be accomplished by a variety of methods including homogenization, acoustic mixing, and high RPM mixing. Depending on the batch size, the size of the mixer, and the type of mixing, the mixing may be conducted for several minutes or more. When mixed in accordance with the parameters disclosed above, the melted petrolatum and the heated PHMB solution fuse in the melted mixture.

After the melted petrolatum and the heated PHMB solution have fused, they are allowed to cool and solidify into the composition described more fully in Section (I) (“the final composition”). Cooling may be achieved by reducing the amount of heat provided to the melted mixture, or cooling may be achieved passively under conditions where no heating is added. In some embodiments, cooling is controlled so that the temperature of the melting mixture is gradually lowered to ambient temperatures. The product is preferably packaged a few degrees above its solidification point so that the packaging can be filled by pouring the melted mixture. The composition preferably solidifies to the final composition in the package. The package is sealed after this solidification.

The process may be conducted with two or more cationic biocides. The cationic biocides may be dissolved in solvent separately or may be dissolved in the same solvent. Addition of additional cationic biocides does not change the process steps above.

### III. Methods of Use

In another aspect, the invention encompasses a method of preventing or treating inflammatory skin disease in a subject using the compositions described herein.

The compositions may be applied topically to the skin of a subject in need. Subjects in need may be those having or exhibiting one or more inflammatory skin diseases, a pro-inflammatory cytokine response, skin inflammation, or eczema. In certain embodiments, the subject may also exhibit or be expected to have the portion of skin in need of treatment colonized by one or more yeast, fungus, bacteria, or other microorganism, that may be susceptible to treatment by the presently disclosed compositions and methods. The subject is preferably human but the composition may also be useful in animals, particularly mammals, for example domestic animals, livestock, or other types of animals.

Typically, the composition is applied to the skin of the subject. The presently disclosed compositions may also be used as a topical dressing to the skin of a subject in order to prevent or reduce the occurrence of the inflammatory skin disease or inflammatory response. As used herein, the terms “applied to the skin” or “applying to the skin,” in all their forms, as used throughout this disclosure in reference to applying the presently disclosed compositions to the skin of a subject, refers to all modes of administration of the compositions to the skin, portion thereof, and/or nearby skin of a patient including topical administration of the compositions directly to the skin or surrounding skin of a subject or causing contact between the compositions and the skin of a subject through, for instance, a wrap, gauze, or bandage impregnated or containing the presently disclosed compositions.

The amount of composition applied in the methods described herein can and will vary depending on the condition being treated and the severity of that condition. Generally, the amount used is sufficient to cover the affected skin area with a thin layer of the composition. The composition is applied directly to the skin. In some embodiments, the composition is spread so that it forms a thin layer over the treatment area. In other embodiments, the composition is spread by a melting action that occurs as the warmth of the patient's skin and surrounding skin melts the petrolatum or pharmaceutically acceptable carrier. The composition may be covered with a bandage after application. The compositions may also be impregnated into a bandage or other material that is applied to the treatment area.

The composition when applied to the skin portion and surrounding skin is non-irritating and non-cytotoxic. These properties allow the composition to be used on sensitive treatment areas. These characteristics also allow for use to treat or prevent the inflammatory skin disease over a long period, such as for example 2 weeks, 4 weeks, 6 weeks, 8 weeks, or longer without irritation to the treated area. It will be recognized however, that the compositions may be used for shorter periods of time if necessary.

The compositions are also capable of extended release of the PHMB or cationic biocides to the area of application. “Extended release” as used herein means that the compositions release PHMB or cationic biocides to the application site over a period of time extending past twelve hours. The time over which the extended release is provided is variable depending on the amount of the composition that is applied, but in general, the release of PHMB and optionally cationic biocides is extended beyond the initial application and PHMB and cationic biocides has been shown to be released for up to 1 week. This extended release allows the composition to be applied less frequently and improves patient compliance with the treatment.

The compositions of the present disclosure also offer kinetic release when applied to the skin. Kinetic release means that PHMB and other optional anti-fungal agents are released to the treatment area more rapidly when the treatment area is hotter.

### EXAMPLES

The following examples are included to demonstrate preferred embodiments of the disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1. Exemplary Formulation Process

“Formulation 1” was prepared by adding 2540.3 pounds of white petrolatum to a tank that has been cleaned and sterilized in accordance with SOP protocol. In the tank was used to heat the petrolatum to 110° C. to 113° F. to melt the petrolatum. In a separate clean and sanitized container 133.70 pounds of water and the desired amount of BZK and PHMB were added and heated to 122° F. When both phases were at temperature, the solution phase was slowly added to the petrolatum with mixing. The heat was decreased slowly to 96 to 104° F. The product was tested for quality control and transferred to polypropylene drums. The resulting composition was shiny and white to slightly yellow in appearance. Specific gravity at 25° C. matches specification when it is from 0.830-0.910. Viscosity at @ 25° C. TF @ 10 rpm matches specification when it is from about 225,000-300,000 cps. The final formulation contained the following ingredients by weight percent: 95% petrolatum, 0.13% BZK, 0.2% PHMB, and 4.67% water.

### Example 2. Skin Sensitization Evaluation

A study was conducted on the formulation of Example 1, referred to herein as “Formulation 1” to assess skin sensitization. Patches comprising Formulation 1 were affixed directly to the skin of 53 human study participants representing an age range from 18-63 and five skin types. Table 1 presents the participant demographics. Patches remained in place for 48 hours after the first application. Participants were instructed not to remove the patches prior to their 48 hour scheduled visit. Thereafter, the subjects were instructed to remove patches for 24 hours. This procedure was repeated until a series of nine consecutive, 24 hour exposures had been made three times per week for three consecutive weeks. Test sites were evaluated by trained personnel. Following a 10-14 day rest period, a retest/challenge dose was applied once to a previously unexposed test site. Test sites were evaluated by trained personnel 48 and 96 hours after application. The sites were scored based on the International Contact Dermatitis Research Group scoring scale (Rietschel, Fowler, Ed., Fisher's Contact Dermatitis (fourth ed.). Baltimore, Williams & Wilkins, 1995) as presented in Table 2.

TABLE 1
Participant Demographics.

| | |
|---|---|
| Number of subjects enrolled | 53 |
| Number of subjects completing study | 53 |
| Age Range | 18-63 |
| Sex | Male 13 |
| | Female 40 |
| Fitzpatrick Skin Type* | |
| 1 - always burn, does not tan | 0 |
| 2 - burn easily, tan slightly | 4 |
| 3 - burn moderately, tan progressively | 47 |
| 4 - burn a little, always tan | 2 |
| 5 - rarely burn, tan intensely | 0 |
| 6 - never burn, tan very intensely | 0 |

*Agaghe P, Hubert P. Measuring the skin. (p. 473, table 48.1) Springer-Verlag Berlin Heidelberg, 2004.

TABLE 2
Scoring Scale.

| | |
|---|---|
| 0 | No reaction (negative) |
| 1 | Erythema throughout at least ¾ of patch area |
| 2 | Erythema and induration throughout at least ¾ of patch area |
| 3 | Erythema, induration and vesicles |
| 4 | Erythema, induration and bullae |
| D | Site discontinued |
| Dc | Subject discontinued |

No adverse reactions of any kind were reported during the course of study. Accordingly, Formulation 1 gives no identifiable signs or symptoms of primary irritation or sensitization (contact allergy).

### Example 3. Antimicrobial Efficiency Testing

Antimicrobial efficacy testing was conducted according to USP 51. Five microbes were tested. Each organism was inoculated at an inoculum level of 1×10⁶ colony forming units (CFU) per gram for bacteria or 1×10⁵ CFU per gram for yeast and mold. The inoculated samples were then stored at 20-25° C. for 28 days. The population of each microorganism was determined by plate counting at Day 2, 7, 14, 21, and 28. The plate counts were performed at a 1:10 initial dilution using Modified Letheen Broth as the diluent and plated onto Tryptic Soy and Sabouraud Dextrose agar.

A single application of Formulation 1 gave 100% elimination from day 2 to day 28 for all microbes tested (Table 3). Given the 100% elimination, there was a 4 log reduction in the yeast/mold species and a 5 log reduction in the bacterial species (Table 4). Table 5 is a positive control indicating that the method used recovers 80-100% of the microbe in the absence of Formulation 1. Accordingly, the microbes present in the test sample were eliminated under the tested conditions. The results illustrate the broad spectrum of activity for Formulation 1.

TABLE 3
Preservative Testing.

| | Colony Forming Units/gram | | | | | |
|---|---|---|---|---|---|---|
| Organism | Inoculum/g | Day 2 | Day 7 | Day 14 | Day 21 | Day 28 |
| Staphylococcus aureus | 1 × 10⁶ | <10 | <10 | <10 | <10 | <10 |
| (bacteria) ATCC#6538) | | | | | | |
| Pseudomonas aeruginosa | 1 × 10⁶ | <10 | <10 | <10 | <10 | <10 |
| (bacteria) (ATCC#9027) | | | | | | |
| Escherichia coli (bacteria) | 1 × 10⁶ | <10 | <10 | <10 | <10 | <10 |
| (ATCC#8739) | | | | | | |
| Candida albicans (yeast) | 1 × 10⁵ | <10 | <10 | <10 | <10 | <10 |
| (ATCC#10231) | | | | | | |
| Aspergillus niger (mold) | 1 × 10⁵ | <10 | <10 | <10 | <10 | <10 |
| (ATCC#16404) | | | | | | |

TABLE 4
Log Reduction Calculation from Initial Inoculum.

| | 14 days | 28 days |
|---|---|---|
| Aspergillus niger | 4.00 | 4.00 |
| Candida albicans | 4.00 | 4.00 |
| Pseudomonas aeruginosa | 5.00 | 5.00 |
| Escherichia coli | 5.00 | 5.00 |
| Staphylococcus aureus | 5.00 | 5.00 |

TABLE 5
Preservative Testing Validation.

| | | | Microbial | | Percent |
|---|---|---|---|---|---|
| Organism | Inoculum | Dilution | Recovery | Diluent | Recovery |
| Staphylococcus aureus | 76 cfu/plate | 1:10 | 69 cfu/plate | LB | 87% |
| Pseudomonas aeruginosa | 83 cfu/plate | 1:10 | 81 cfu/plate | LB | 97% |
| Escherichia coli | 68 cfu/plate | 1:10 | 58 cfu/plate | LB | 85% |
| Candida albicans | 63 cfu/plate | 1:10 | 50 cfu/plate | LB | 79% |
| Aspergillus niger | 60 cfu/plate | 1:10 | 60 cfu/plate | LB | 100% |

CFU = colony forming units;
LB = Letheen Broth;
Diluent = Letheen Broth;
Dilution: 1:10

Example 3 indicates that the presently disclosed compositions are effective against Candida albicans a common causative agent of skin colonization and infestations.

### Example 4: Cytotoxicity Evaluation

The study was conducted to assess the biological reactivity of mammalian cells (grown in culture) to the agar-diffusible elements of Formulation 1.

The samples to be evaluated for cytotoxicity include test product comprising Formulation 1, Amber latex tubing as a positive control, and HDPE sheet stock as a negative control. The samples were sized to have no less than 100 mm² of contact surface and provide coverage of approximately 10% of the test dish. The dimensions of the test product comprising Formulation 1 were 1.1×1.1-1.2 cm; the dimensions of the positive control were 1.0×2.55-2.7 cm; and the dimensions of the negative control were 1.15×1.0-1.2 cm. The manipulation of the samples was performed aseptically.

Prior to exposure to the samples, the L929 Mouse Fibroblast cells were subcultured in Minimum Essential Medium (MEM) with 10% Fetal Bovine Serum (FBS) to achieve a confluency of approximately 80+10% at the time of exposure. The cells were examined for normal morphology and the absence of contamination. Once the cells met the acceptance criteria for use, individual dishes were numbered in triplicate to represent the controls and the test product comprising Formulation 1.

On the day of testing, the subculture media was carefully removed from each test dish and replaced with a 2 mL aliquot of the 1:1 overlay medium (in equal parts of 2× Minimum Essential Medium (with 2% Fetal Bovine Serum) and Agar Noble). After allowing the overlay medium to solidify, a single test product comprising Formulation 1 or control sample was placed in the center of each dish (in contact with the agar surface). Triplicate cultures were prepared for each test product comprising Formulation 1 and positive and negative controls (one sample per dish). When the test product comprising Formulation 1 or positive/negative control has only one face designated for patient-contact, that “side” of the sample was directed toward the agar. The test dishes, along with 3 dishes with overlay medium only (Monolayer Negative Controls), were then placed in the 37° C./5% CO₂ incubator to initiate the exposure interval.

The dishes were incubated for 24 hours and then microscopically examined for an indication of cellular response. A preliminary microscopic examination of the cells was made prior to staining and before the control and test product comprising Formulation 1 were removed from the agar layer. The cells were then stained with a fresh working Neutral Red Solution to facilitate response grading. The test product comprising Formulation 1 and control samples were removed from the dishes at this time. The stained cells were then fixed by the addition of buffered formalin. Following fixation, the agar overall was removed from each dish. Following staining, the cellular responses were then evaluated microscopically and macroscopically (by examining the dishes against a white surface) and the results were recorded.

For the control samples to be deemed valid, the negative controls may be no greater than Grade 0 and the positive control may be no less than Grade 3. For the test product comprising Formulation 1, a Grade of 0, 1 (slight) or 2 (mild) indicates the test product comprising Formulation 1 “meets” the assay acceptance criteria and a Grade of 3 (moderate) or 4 (severe) indicates the test product comprising Formulation 1 does not meet the assay acceptance criteria. Table 6 depicts the Grading guidelines.

TABLE 6
Grading Guidelines.

| Grade(1) | Reactivity | Description of the Reactivity Zone(2) |
|---|---|---|
| 0 | None | No detectable zone around or under specimen |
| 1 | Slight | Some malformed or degenerated cells |
| | | under specimen(3) |
| 2 | Mild | Zone limited to area under specimen |
| 3 | Moderate | Zone extends 0.45 to 1.0 cm beyond specimen |
| 4 | Severe | Zone extends greater than 1.0 cm |
| | | beyond specimen |

(1)The use of the above Grading Table is contingent on the test article meeting the minimum surface area requirements of ≥100 mm². Should samples of smaller dimensions be tested, the reactivity (if any) would be expected to be less and the grading would need to be justified.
(2)The extent of the Reactivity Zone is the maximum measured distance from the edge of the specimen to the margin of monolayer where degenerated cells are no longer observed. Where described as “under specimen”, this maximum measured distance is limited to <0.45 cm beyond the specimen.
(3)To be interpreted as “slight” reactivity, no more than 50% of the cells under the specimen may exhibit reactivity as rounding and/or lysis.

Table 7 depicts the results of the study. The assay controls met the acceptance criteria for a valid assay. All negative controls responses were no greater than Grade 0 and the positive control response were not less than Grade 3. The responses observed for the test product comprising Formulation 1 were interpreted according to the current USP guidelines. The Grade 1 response from the test product comprising Formulation 1 is considered to be “non-cytotoxic” (i.e. meets ISO test acceptance requirements of no more than Grade 2 reactivity). Accordingly, Formulation 1 does not damage mammalian cells.

TABLE 7
Study Results.

| | | Macroscopic Reading | Microscopic Reading | |
|---|---|---|---|---|
| | | (Zone Dimensions) | (% Rounded/Lysed) | Grade |
| Monolayer Negative | 1 | No detectable zone | 0%/0% | 0 |
| Control | 2 | No detectable zone | 0%/0% | 0 |
| | 3 | No detectable zone | 0%/0% | 0 |
| Material Positive | 1 | Clear Zone 3.2 × 3.2; | 100%/100% | 4 |
| Control | | Greatest distance from | | |
| | | specimen 1.5 cm | | |
| | 2 | Clear Zone 3.2 × 3.2; | 100%/100% | 4 |
| | | Greatest distance from | | |
| | | specimen 1.5 cm | | |
| | 3 | Clear Zone 3.2 × 3.2; | 100%/100% | 4 |
| | | Greatest distance from | | |
| | | specimen 1.5 cm | | |
| Material Negative | 1 | No detectable zone | 0%/0% | 0 |
| Control | 2 | No detectable zone | 0%/0% | 0 |
| | 3 | No detectable zone | 0%/0% | 0 |
| Test Product | 1 | Entire dish lightly | 1.5%/1.5% | 1 |
| Comprising | | stained ~5% rounded | | |
| Formulation 1 | | directly under sample | | |
| | 2 | Entire dish lightly | 1.5%/1.5% | 1 |
| | | stained ~5% rounded | | |
| | | directly under sample | | |
| | 3 | Entire dish lightly | 1.5%/1.5% | 1 |
| | | stained ~5% rounded | | |
| | | directly under sample | | |

### Example 5: Rabbit Skin Irritation

The study was conducted to assess the irritating potential of Formulation 1 to produce dermal irritation.

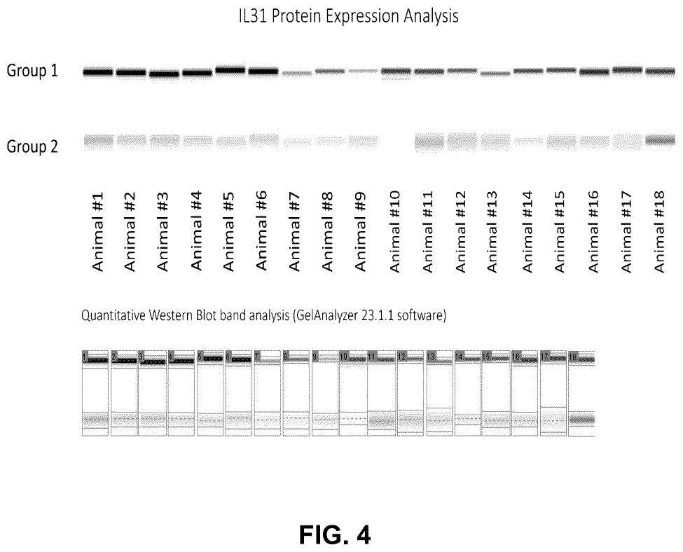

Within 24 hours to 4 hours before test application, the backs of female albino New Zealand White rabbits were clipped free of hair, exposing 2 test and 2 control areas on each side of the spine with a size of approximately 15 cm×15 cm. The two test sites are located on the left cranial section and the right caudal section of the dorsal region. The two control sites are located on the left caudal and right cranial section of the dorsal region. FIG. 4 depicts the arrangement of test and control sites. The exposed skin is wiped with alcohol and dried. Rabbits of acceptable skin quality were selected and used for testing.

A 25×25 mm gauze patch saturated with 0.5 mL (liquid) or 0.5 g (powder) of Formulation 1 is applied to the clipped test sites. A 25×25 mm gauze patch saturated with 0.5 mL of 0.9% NaCl is used for the control and applied to the clipped control sites. The patches are secured using hypoallergenic, waterproof, surgical tape over the test and control sites. The animal's trunk is securely wrapped so as to maintain the position of the patches. Patches are left applied for a minimum of four hours.

After patch removal, the test and control sites were then scored for erythema and edema at 1, 24, 48 and 72 hours after patch removal. Only the 24, 48, and 72 hour observations were scored and used for calculations. The criteria for scoring is presented in Table 8. If no response was expected, testing was conducted using three animals per test article. If irritation was anticipated, one animal was tested initially. If the first animal received a score of 2 or less for either erythema or edema, 2 additional rabbits were used to conclude the test.

TABLE 8
Scoring Criteria for Test Reactions.

| Reaction | Description | Score |
|---|---|---|
| Erythema (ER) | No erythema | 0 |
| | Very slight (barely perceptible) | 1 |
| | Well defined | 2 |
| | Moderate | 3 |
| | Severe (beet-redness) to eschar | 4 |
| | formation preventing grading of erythema | |
| Edema (ED) | No edema | 0 |
| | Very slight (barely perceptible) | 1 |
| | Well-defined edema (edges of area | 2 |
| | well-defined by definite raising | |
| | Moderate (edges raised ~1 mm) | 3 |
| | Severe (raised more than 1 mm and | 4 |
| | extending beyond exposure area) | |

For each animal and each extract, when applicable, the scores for the test article comprising Formulation 1 for erythema and edema at each time were added. This total was divided by the total number of observations. The same was done for the control sites. The control result was subtracted from the test results to give the irritation index for each animal. These scores for each animal were added and divided by the total number of animals to give the Primary Irritation Index. The Primary Irritation Index is depicted in Table 9. For any response, the Maximum Irritation Response, the time of onset of the response and the time of maximum response was recorded.

TABLE 9
Primary Irritation Index

| Primary Irritation Index | Response Category |
|---|---|
| 0-0.4 | Negligible |
| 0.5-1.9 | Slight |
| 2-4.9 | Moderate |
| 5-8 | Severe |

The results indicated that the skin reactions for both the test article comprising Formulation 1 and control samples were not significant. That data is presented in Table 10 below. Accordingly, the Formulation 1 is non-irritating.

TABLE 10
Direct Application of Test Article.

| | Formulation 1 | Control |
|---|---|---|
| Rabbit | ER + ED = Total | ER + ED = Total |
| No. 14384 | 0 0 0 | 0 0 0 |

Test Total − Control Total = 0
Total Score Average = 0

| | | |
|---|---|---|
| Rabbit | ER + ED = Total | ER + ED = Total |
| No. 14387 | 0 0 0 | 0 0 0 |

Test Total − Control Total = 0
Total Score Average = 0

| | | |
|---|---|---|
| Rabbit | ER + ED = Total | ER + ED = Total |
| No. 14394 | 0 0 0 | 0 0 0 |

Test Total − Control Total = 0
Total Score Average = 0

Total Average (0) = 0 Primary Irritation Index
No. of Animals (3)

To positively validate the test, 10% sodium dodecyl sulfate (SDS), which is a known dermal irritant, in petroleum jelly was applied to a 2.5 cm×2.5 cm gauze patch. As a negative control, 0.5 mL of 0.9% NaCl was applied to a 2.5 cm×2.5 cm gauze patch. A Primary Irritation Index in the moderate to severe range is considered a positive result. The test system and methods utilized were the same as described above. Table 11 presents the results validating the study.

TABLE 11
Primary Skin Positive Validation Test.

| | 10% SDS | Control |
|---|---|---|
| Rabbit | ER + ED = Total | ER + ED = Total |
| No. 14279 | 18 14 32 | 0 0 0 |

Test Total − Control Total = 32
Total Score Average = 5.3

| | | |
|---|---|---|
| Rabbit | ER + ED = Total | ER + ED = Total |
| No. 14280 | 21 19 40 | 0 0 0 |

Test Total − Control Total = 40
Total Score Average = 6.6

| | | |
|---|---|---|
| Rabbit | ER + ED = Total | ER + ED = Total |
| No. 14281 | 23 21 44 | 0 0 0 |

Test Total − Control Total = 44
Total Score Average = 7.3

Total Average (19.3) = 6.4 Primary Irritation Index
No. of Animals (3)

### Example 6. Suspension Time-Kill Procedure for MRSA, T. rubrum, and Staphylococcus epidermidis

A study was conducted to evaluate the changes in the population of MRSA in an antimicrobial liquid suspension comprising Formulation 1. Methicillin-resistant Staphylococcus aureus (MRSA) is a Gram-positive, cocci shaped, aerobe which is resistant to the penicillin-derivative antibiotic methicillin. MRSA can cause troublesome infections, and their rapid reproduction and resistance to antibiotics makes them more difficult to treat. MRSA bacteria are resistant to drying and can therefore survive on surfaces and fabrics for an extended period of time and therefore makes this bacteria, an excellent representative for antimicrobial efficacy testing on surfaces.

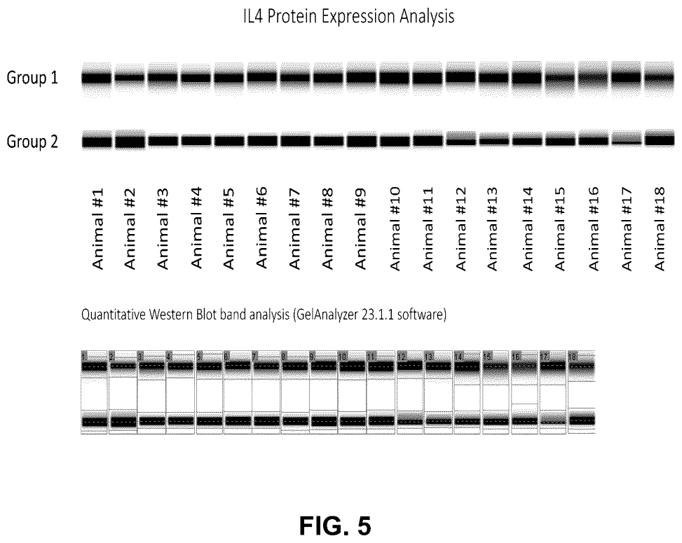

To conduct the study, MRSA was prepared in liquid culture medium (Letheen Broth). The suspension of MRSA was standardized by dilution to 10⁶ in a buffered saline solution. Formulation 1 and control substance (PBS) were dispensed in identical volumes to sterile vessels. Independently, Formulation 1 and control substance were each inoculated with MRSA, then mixed and incubated. Control substances were immediately harvested and represented the concentration present at the start at the test (i.e. time zero). At the conclusion of contact time, a volume of the liquid test product was harvested and chemically neutralized. Dilutions of the neutralized test solution were assayed using appropriate growth media to determine the surviving MRSA at the respective contact times. Reductions in MRSA were calculated by comparing initial microbial concentrations to final microbial concentrations. Table 12 and FIG. 5 present the results of the study.

TABLE 12
Results of Suspension Time-Kill Test for MRSA (33592)

| | | | | | Percent | Log₁₀ |
|---|---|---|---|---|---|---|
| | | | | | Reduction | Reduction |
| | | | | | vs. Control | vs. Control |
| Test | Contact | | Replicate | Average | at Time | at Time |
| substance | time | Replicate | CFU/ml* | CFU/ml | Zero | Zero |
| PBS | Time | 1 | 1.75E+06 | 1.48E+06 | N/A | |
| | Zero | 2 | 1.20E+06 | | | |
| Formulation 1 | 30 seconds | 1 | 1.00E+01 | <1.00E+01 | >99.9993% | >5.17 |
| | | 2 | <1.00E+01 | | | |
| | 2 minutes | 1 | 1.00E+01 | <1.00E+01 | >99.9993% | >5.17 |
| | | 2 | <1.00E+01 | | | |

*The limit of detection for the assay is 1.00E+01 CFU/ml. Values below the limit of detection are notes as <1.00E+01 in the table.

The same study was conducted with Trichophyton rubrum. T. rubrum is a fungus which belongs to the dermatophyte group. Dermatophytes commonly cause skin disease in animals and humans. T. rubrum is anthropophilic, meaning it preferentially infects humans over animals. This parasite is the most common cause of fungal infection of the fingernail and Athlete's foot, this specific strain was isolated from a human toenail. In the laboratory, visible colonies can be observed after approximately 4-5 days and are fluffy and white in appearance. T. rubrum is a popular test microorganism for fungicidal testing, especially for products intended for use in environments where skin infections can occurs and spread rapidly such as locker rooms and schools.

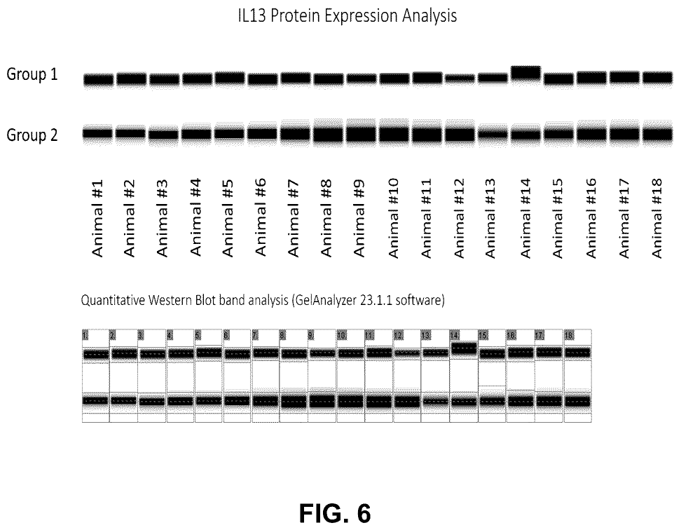

To conduct the study, T. rubrum was prepared on agar (potato dextrose agar). The T. rubrum was resuspended and inoculated at a dilution of ˜10⁶ into vessels containing Formulation 1 and control substance (PBS). Control substances were immediately harvested and represented the concentration present at the start at the test (i.e. time zero). At the conclusion of contact time (2 or 10 minutes), a volume of the liquid test product was harvested and chemically neutralized. Dilutions of the neutralized test solution were assayed using appropriate growth media to determine the surviving T. rubrum at the respective contact times. Reductions in T. rubrum were calculated by comparing initial microbial concentrations to final microbial concentrations. Table 13 and FIG. 6 present the results of the study.

TABLE 13
Results of Suspension Time-Kill Test for T. rubrum (MYA-4438)

| | | | | | Percent | Log₁₀ |
|---|---|---|---|---|---|---|
| | | | | | Reduction | Reduction |
| | | | | | vs. Control | vs. Control |
| Test | Contact | | Replicate | Average | at Time | at Time |
| substance | time | Replicate | CFU/ml* | CFU/ml | Zero | Zero |
| PBS | Time | 1 | 2.55E+05 | 3.15E+05 | N/A | |
| | Zero | 2 | 3.75E+05 | | | |
| Formulation 1 | 2 minutes | 1 | 3.50E+03 | 2.18E+03 | 99.31% | 2.16 |
| | | 2 | 8.50E+02 | | | |
| | 10 minutes | 1 | <5.00E+01 | <5.00E+01 | >99.98% | >3.80 |
| | | 2 | 5.00E+01 | | | |

*The limit of detection for the assay is 5.00E+01 CFU/ml. Values below the limit of detection are notes as <5.00E+01 in the table.

The same study was conducted with Staphylococcus epidermidis. Gram-positive organisms currently account for 50-60% of nosocomial bacteremic events. Staphylococcus epidermidis is the most common gram-positive organism isolated from blood (30% of isolates) and accounts for the majority of infections that are associated with intravascular catheters, as it is capable of forming antibiotic resistant biofilms on plastic surfaces.

In an effort to further explore the preventative benefits of Formulation 1 in preventing catheter related and hospital acquired infections, a suspension time kill assay as described above was initiated on this often under-discussed organism. A nearly 7 log kill over 24 hours was observed, which represents a typical change interval for intravenous catheter dressings (Table 14).

TABLE 14
Results of Suspension Time-Kill Test for S. epidermidis (ATCC 12228)

| | | | | Log₁₀ Reduction vs. |
|---|---|---|---|---|
| Test | | Replicate | Percent Reduction vs. | Control at Time |
| substance | Contact time | CFU/ml* | Control at Time Zero | Zero |
| PBS | Time Zero | 4.80E+06 | N/A | |
| Formulation 1 | 24 Hours | <1.00E+00 | >99.99998% | >6.68 |

The limit of detection is 1.00E+00 and is represented as <1.00E+00.

Example 6 indicates that the presently disclosed compositions are effective against Trichophyton rubrum a common causative agent of skin colonization infestations. Specifically, after 10 minutes, the Formulation 1 composition of Example 1 reduced T. rubrum by more than 99.98%.

### Example 7. Stability

Formula I as packaged in tubes was subjected to an accelerated stability study. Formula I was placed sideways in a 40° C.±2° C./75%±5% relative humidity (RH) storage chamber for different intervals to yield a period of three months. The product was assessed for physical and analytical characteristics. When stored at 40° C.±2° C./75%±5% (RH)benzyl alkonium chloride was stable as shown in Table 15.

TABLE 15
Accelerated Stability Testing

| Analytical | | Initial: | 1 Month | 2 Months | 3 Months |
|---|---|---|---|---|---|
| Assay Testing | Specification | Assessing | Assessing | Assessing | Assessing |
| Benzalkonium | 0.0071%-0.0086% | 0.0084% | 0.0085% | 0.0075% | 0.0086% |
| Chloride | | | | | |
| 0.0081% | | | | | |

Additionally, the product met specification for appearance, odor, specific gravity, viscosity and package compatibility at all time points tested.

Formula I was also tested under for microbial counts at 40° C.=2° C./75%=5% were as shown below. The results are shown in Table 16.

TABLE 16
Accelerated Stability Testing

| | | | Results | | | |
|---|---|---|---|---|---|---|
| | | | Initial | 1 Month | 2 Months | 3 Months |
| Micro Testing | SPEC | Method | Assessing | Assessing | Assessing | Assessing |
| Total Plate Count | <100 | TM-01 | <10 | <10 | <10 | <10 |
| (TPC) | cfu/ml | | cfu/ml | cfu/ml | cfu/ml | cfu/ml |
| Yeast/Mold | <100 | TM-01 | <10 | <10 | <10 | <10 |
| | cfu/ml | | cfu/ml | cfu/ml | cfu/ml | cfu/ml |
| Enrichment | Absent | Absent | Absent | Absent | Absent | Absent |
| (Pathogens) | | | | | | |
| Pseudomonas | Absent | Absent | Absent | Absent | Absent | Absent |
| S. aureus | Absent | Absent | Absent | Absent | Absent | Absent |
| E. coli | Absent | Absent | Absent | Absent | Absent | Absent |
| Coliforms | Absent | Absent | Absent | Absent | Absent | Absent |
| Salmonella/Shigella | Absent | Absent | Absent | Absent | Absent | Absent |

Additionally, the product met specification for appearance, odor, specific gravity, viscosity and package compatibility at all time-points tested when under standard conditions for over nine months.

### Example 8. Formulation Example 1

A petrolatum-based composition in accordance with the present disclosure was prepared by mixing 1000 cc of water containing 2% by weight PHMB and 0.13% by weight BZK. The water solution was heated to 40° C. and was then added to 19,000 cc of petrolatum at about 45° C. to give 20,000 cc of a petrolatum-based composition containing BZK and PHMB.

### Example 9. Formulation Example 2

A petrolatum-based composition in accordance with the present disclosure was prepared by mixing 1000 cc of water containing 2% by weight PHMB. This solution was then added to 20,000 cc of petrolatum to give 21,000 cc of a petrolatum-based composition containing PHMB.

### Example 10. Formulation Example 3

A petrolatum-based composition in accordance with the present disclosure was prepared by mixing PHMB, BZK and water to form a PHMB solution that contained 10% by weight PHMB, 0.13% by weight BZK, and 89.87% by weight water. The PHMB solution was heated to a temperature from about 1° C. to about 5° C. higher than the temperature of the melted petrolatum and mixed to form a composition comprising 95% by weight petrolatum, 4.4935% by weight water, 0.5% by weight PHMB, and 0.0065% by weight BZK.

### Example 11. Formulation Example 4

A petrolatum-based composition in accordance with the present disclosure was prepared by mixing 100 mL of a 20% PHMB solution, 162.5 mL of a 80% BZK solution, and 737.5 mL of water to form 1000 mL of a PHMB solution. Separately, 1900 mL of petrolatum was heated to a temperature sufficient to completely melt the petrolatum and left at that temperature. The 1000 mL PHMB solution was then heated to a temperature from about 1° C. to about 5° C. higher than the temperature of the melted petrolatum and mixed with the 19000 mL heated petrolatum and stirred to form a melted mixture of PHMB and petrolatum. The melted mixture was then allowed to cool to room temperature to form the petrolatum-based PHMB composition.

### Example 12. Formulation Example 5

A petrolatum-based composition in accordance with the present disclosure was prepared by mixing 100 mL of a 20% PHMB solution, 162.5 mL of a 80% BZK solution, 100 mL of ethanol, and 637.5 mL of water to form 1000 mL of a PHMB solution. Separately, 1900 mL of petrolatum was heated to a temperature sufficient to completely melt the petrolatum and left at that temperature. The 1000 mL PHMB solution was then heated to a temperature from about 1° C. to about 5° C. higher than the temperature of the melted petrolatum and mixed with the 19000 mL heated petrolatum and stirred to form a melted mixture of PHMB and petrolatum. The melted mixture was then allowed to cool to room temperature to form the petrolatum-based PHMB composition.

### Example 13. Formulation Example 6

A petrolatum-based composition in accordance with the present disclosure was prepared by mixing 100 mL of a 20% PHMB solution, 162.5 mL of a 80% BZK solution, 200 mL ethanol, and 537.5 mL of water to form 1000 mL of a PHMB solution. Separately, 1900 mL of petrolatum was heated to a temperature sufficient to completely melt the petrolatum and left at that temperature. The 1000 mL PHMB solution was then heated to a temperature from about 1° C. to about 5° C. higher than the temperature of the melted petrolatum and mixed with the 19000 mL heated petrolatum and stirred to form a melted mixture of PHMB and petrolatum. The melted mixture was then allowed to cool to room temperature to form the petrolatum-based PHMB composition.

### Example 14. Efficacy of Formulation 1 of Example 1 on in vivo Staphylococcus aureus dermal efficacy and toxicity study

A study was conducted on the formulation of Example 1, referred to herein as “Formulation 1” to assess efficacy to treat or control S. aureus in the skin of a subject as well as to assess dermal toxicity of the presently disclosed formulations and compositions. A non-GLP study was used to preclinically evaluate the in vivo therapeutic efficacy of topical administration of Formulation 1 in an animal model of S. aureus epicutaneous exposure and its effect on skin inflammation via IL-36 mediated T cell responses. Thirty (30) female mice were obtained and went through mandatory 1-week acclimatization per IACUC guidelines. All animal procedures and maintenance were conducted in accordance with the institutional guidelines. The administration of the test articles and the animal numbers in each study group are shown in the following experimental design table.

TABLE 17
Experimental Design

| | Number of | | Dosing | S. aureus | Treatment |
|---|---|---|---|---|---|
| Group | Mice | Treatment | Route | Colonization | Schedule |
| 1 | 10 | Pretreatment | NA | Yes | NA |
| | | Control | | | |
| 2 | 10 | No | NA | Yes | NA |
| | | Treatment | | | |
| | | Control | | | |
| 3 | 10 | Test | Topical | Yes | Twice Daily |
| | | Compound | | | |

Staphylococcus aureus bacteria were obtained from ATCC. Bacteria were grown on a tryptic soy agar (TSA) plate (tryptic soy broth [TSB] plus 1.5% bacto agar (BD) and grown overnight at 37° C. in a bacterial incubator. Single colonies were picked and cultured in TSB at 37° C. in a shaking incubator (240 rpm) overnight (18 h), followed by a 1:50 subculture at 37° C. for 2 h to obtain mid-logarithmic phase bacteria. The bacteria were pelleted, washed 3 times, and resuspended in sterile PBS at a concentration of 1×10⁸ CFU/100 μL. The absorbance (A600) was measured to estimate the number of CFU, which was verified after overnight culture on TSA plates. A 100 μL volume of PBS containing 1×10⁸ CFU of Staphylococcus aureus bacteria was placed on a sterile gauze pad (1×1 cm) and attached to the skin with dressing (Tegaderm; 3M), and secured with 2 layers of adhesive bandages (BAND-AID, Johnson and Johnson) for 7 days. The severity of skin inflammation was assessed by digital photographs and quantified using a total disease score (see assay criteria below) by performing established by validated investigator global assessment scale for Atopic Dermatitis (vIGA-ADTM):

TABLE 18
Validated Investigator Global Assessment Scale
for Atopic Dermatitis vIGA-AD ™

| Score | Morphological Description |
|---|---|
| 0 - Clear | No inflammatory signs of atopic dermatitis (no |
| | erythema, no induration/papulation, no |
| | lichenification, no oozing/crusting). Post- |
| | inflammatory hyperpigmentation and/or |
| | hypopigmentation may be present. |
| 1 - Almost Clear | Barely perceptible erythema, barely |
| | perceptible induration/papulation, and or |
| | minimal lichenification. No oozing or |
| | crusting. |
| 2 - Mild | Slight but definite erythema (pink), slight but |
| | definite induration/papulation, and/or slight |
| | but definite lichenification. No oozing or |
| | crusting. |
| 3 - Moderate | Clearly perceptible erythema (dull red), clearly |
| | perceptible induration/papulation, and/or |
| | clearly perceptible lichenification. Oozing or |
| | crusting may be present. |
| 4 - Severe | Marked erythema (deep or bright red), marked |
| | induration/papulation, and/or marked |
| | lichenification. Disease is widespread in |
| | extent. Oozing or crusting may be present. |

The IGA score is selected using the descriptors in Table 18 that best describe the overall appearance of the lesions at a given time point. It is not necessary that all characteristics under the morphological description in Table 18 be present. After 7 days of Staphylococcus aureus skin inflammation model treatment, 9 mice were euthanized by CO₂ asphyxiation and skin tissue dissected, washed twice in 1×PBS and blot dry with a kim wipe and placed into 400 μl of lysis buffer (0.01 M PBS, 2% (w/v) SDS, 5 mM EDTA, 1X Halt protease inhibitor). Tissues were homogenized in lysis buffer to form a uniform slurry. Slurry centrifuged at 12,000×g for 10 minutes, supernatant collected and stored at −80° C. until further use in Western Blot analysis. Each sample was used to measure total protein concentration (Bradford assay, Bio-Rad) and concentration of each individual sample normalized (each sample have the same total protein concentration and same volume loaded per each of the gel well).

Normalized samples were loaded on polyacrylamide gel (precast gels 4%-15%, Bio-Rad) and run with constant voltage set at 100 V for 15 min, then 160 V with cold pack and prechilled buffer. PVDF membrane (Millipore Immobion-P) was washed in methanol and transferred into 1× transfer buffer until ready to use. Protein transfer was completed following “sandwich” assemble (per manufacturer's recommended transblot protocol, Bio-Rad).

Transfer preformed for 1 hr with constant current at 360 mA with the cold pack and prechilled buffer. Membrane was immersed in blocking buffer (5% milk in 1×PBS) and blocked for 30 minutes. Antibodies and detection: Membranes incubated with primary antibodies at RT for 60 min (mouse IL-36a antibody, 2 μg/mL blocking buffer, R&D Systems), followed by washing in 0.05% Tween 20 in 1×PBS (3 times x 5 min). Membrane was incubated with secondary antibody (Abcam) for 60 min at room temp), diluted in 2% milk in 1×PBS buffer for 60 min at room temp. Washed 3×5 min with 0.05% Tween 20 in 1×PBS. Signal detection with Western Blue stabilized substrate for alkaline phosphatase (Promega Cat: S3841) following manufacturer recommended protocol. Three out of thirty mice had their gauzes and bandages fall off and were subsequently removed from the study. Nine mice were randomly assigned to each of the three groups.

Summary statistics, the mean and the standard error of the mean (SEM), are provided for each group. Statistical analysis of IL-36 expression among the groups and the analysis of test article interaction were conducted on the experimental data obtained. Western Blot sample analysis performed by GelAnalyzer 23.1.1 software. There was no animal death observed within the study. There were no clinical signs or behavioral phenotypes observed within the study (daily cage intensive observation for adverse effect were performed). No animals were observed to have>20% BWL within the study period.

The IGA score summaries and results of the study are provided below in Tables 19-20 as well as in FIG. 1. As shown in Table 20 and FIG. 1, the IGA score went down from 3.00 for the no treatment group to 1.44 for the formulation 1 treatment group. The decrease was accompanied by a very low p-value indicating that the data are statistically significant at a high confidence level.

TABLE 19
IGA Score Summary Based on Skin Appearance
After 7 Days of S. aureus Treatment

| Animal # | Group 1 | Group 2 | Group 3 |
|---|---|---|---|
| 1 | 4 | 4 | 4 |
| 2 | 3 | 3 | 4 |
| 3 | 3 | 3 | 3 |
| 4 | 2 | 3 | 4 |
| 5 | 4 | 3 | 2 |
| 6 | 3 | 2 | 3 |
| 7 | 3 | 4 | 2 |
| 8 | 4 | 4 | 4 |
| 9 | 4 | 4 | 4 |
| Mean: | 3.33 | 3.33 | 3.33 |

TABLE 20
IGA Score Summary Based on Skin Appearance at 14 Days - 7 Days
of S. aureus Treatment Followed by 7 Days of Negative Control
(Group 2) or 7 Days of Formulation 1 Treatment (Group 3)

| Animal # | Group 2 | Group 3 |
|---|---|---|
| 1 | 4 | 1 |
| 2 | 3 | 2 |
| 3 | 2 | 1 |
| 4 | 2 | 2 |
| 5 | 3 | 1 |
| 6 | 2 | 1 |
| 7 | 4 | 1 |
| 8 | 3 | 2 |
| 9 | 4 | 2 |
| Mean: | 3.00 | 1.44 |

The results of this study indicate that treatment with Formulation 1 reduces disease severity in an atopic dermatitis model by 57% in-vivo in as few as 7 days as compared to a 10% reduction for the placebo.

### Example 15. Efficacy of Formulation 1 of Example 1 on Eczema, Eczema-Like Inflammation, and Cytokine Inhibition

A seven day animal study was conducted to determine the effect of the formulation of Example 1, referred to herein as “Formulation 1” on the pathogenesis of eczema, eczema-like inflammation and cytokine inhibition. In particular, the study is designed to ascertain the immunological inhibitory effects that Formulation 1 has during eczema induction. After a one week acclimatization period, twenty female C57BL/6 mice were pre-treated for 4 days with Formulation 1 (Group 2) and then subsequently colonized using a Staphylococcus aureus induction model for 3 days in order to simulate eczema-like inflammation. A second group of twenty female C57BL/6 mice (Group 1) were colonized using a Staphylococcus aureus induction model for 3 days in order to simulate eczema-like inflammation without first being pretreated with Formulation 1.

The severity of skin inflammation was quantified using a total disease score by performing validated Investigator Global Assessment (IGA) scale scores for Atopic Dermatitis (vIGA-ADTM) for each animal, as shown in Table 18.

A broad array of cytokines were assessed at the skin surface at the end of the 3 day induction period, including IL-4, IL-13, IL-31, IL-36 alpha, and IL-36 gamma. All mice were euthanized by CO₂ asphyxiation and skin tissue dissected (aprox 1 cm×1 cm×1 mm). Dissected skin tissue was washed twice in 1×PBS and blot dry with a kim wipe and placed into 800 μl of lysis buffer (0.01 M PBS, 2% (w/v) SDS, 5 mM EDTA, 1X Halt protease inhibitor). Tissues were homogenized in a lysis buffer to form a uniform slurry. Slurry centrifuged at 12,000×g for 10 minutes, supernatant collected and stored at −80° C. until further use in Western Blot analysis. Each sample was used to measure total protein concentration via Bradford assay (cat #5000201, Bio-Rad) following manufacturer recommended protocol. Concentration of each individual sample were normalized (diluted to) 1 mg/ml, so that each sample have the same total protein concentration and same volume loaded per each of the gel well. Concentration of normalized samples were confirmed via Bradford assay to be within the range of 1.00-1.04 mg/ml.

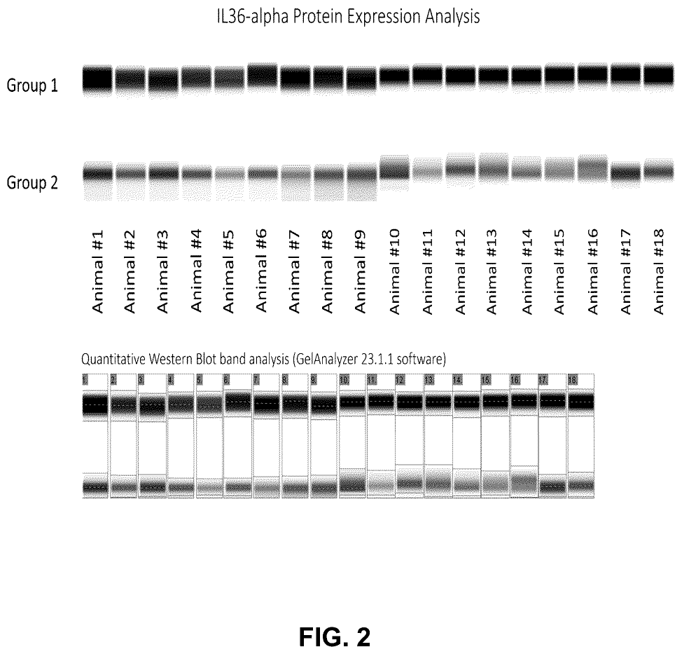

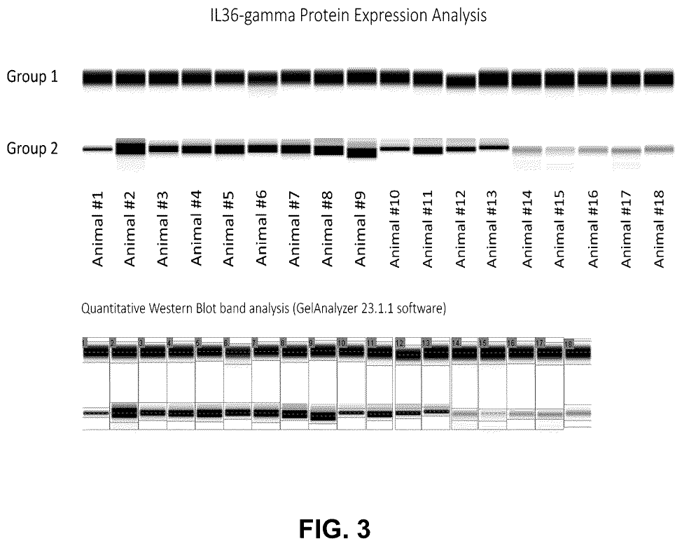

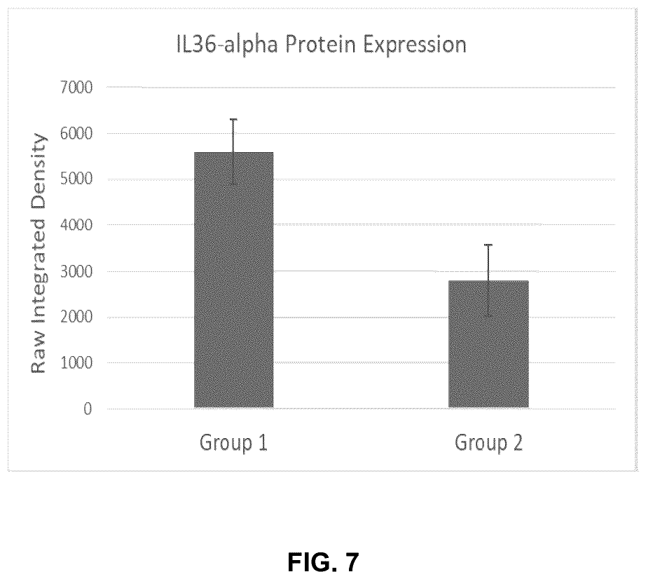

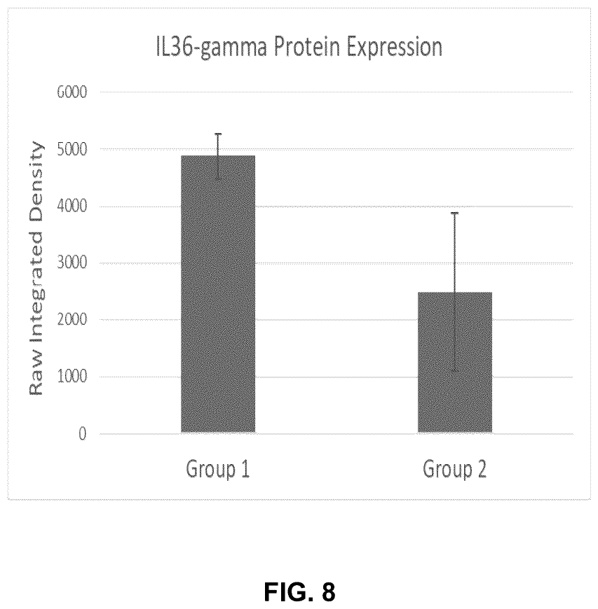

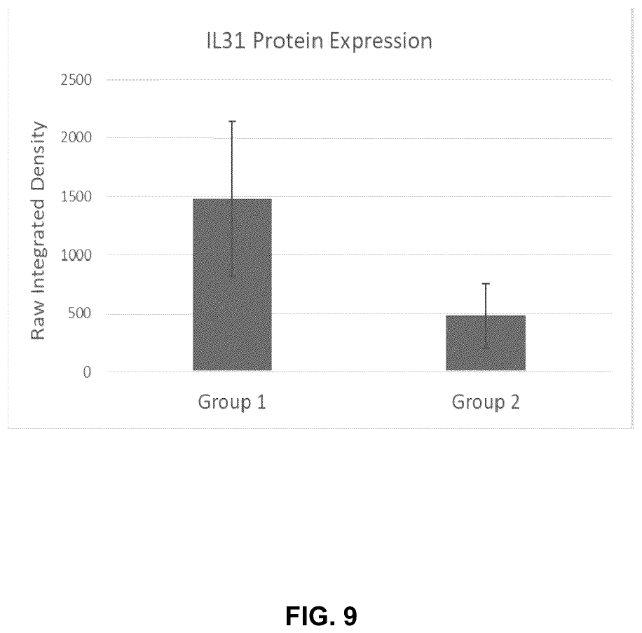

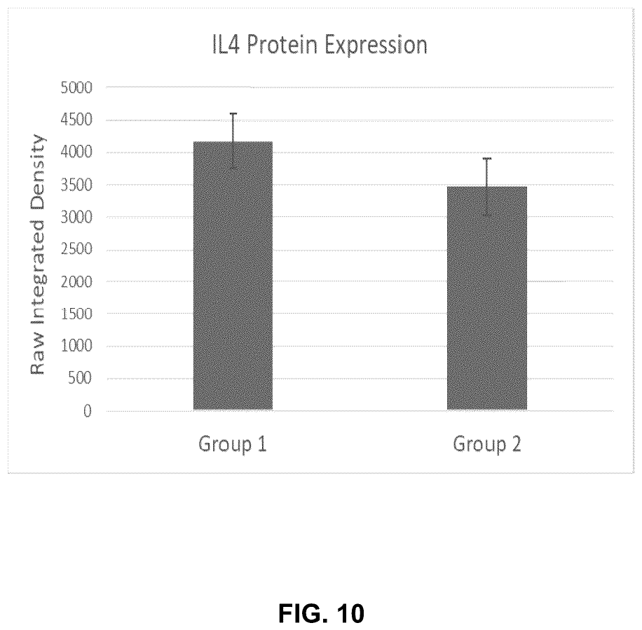

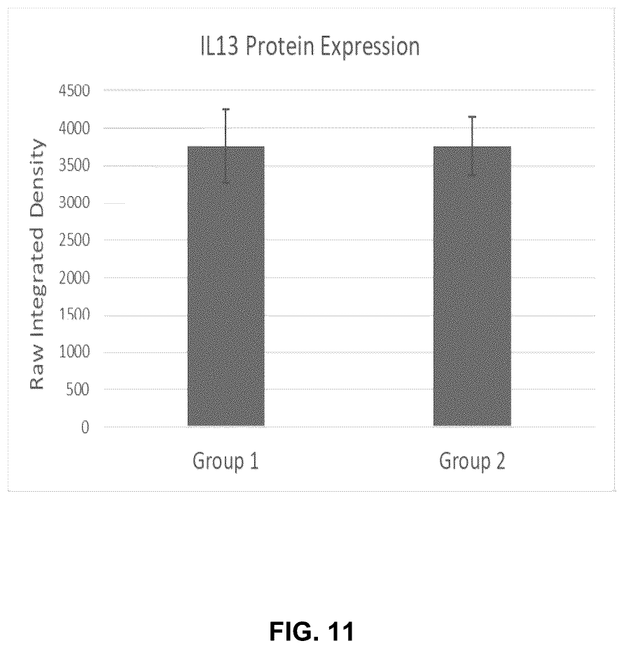

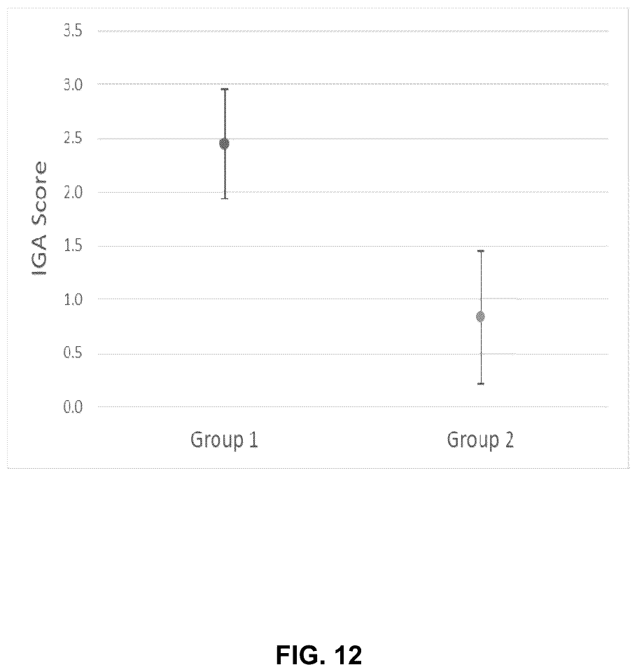

Normalized samples (10 ug in 10 μl volume) were loaded on polyacrylamide gel (precast gels 4%-15%, Bio-Rad) and run with constant voltage set at 100 V for 15 min, then 160 V with cold pack and prechilled buffer. PVDF membrane (Millipore Immobion-P) was washed in methanol and transferred into 1× transfer buffer until ready to use. Protein transfer was completed following “sandwich” assemble (per manufacturer's recommended transblot protocol, Bio-Rad). Transfer preformed for 1 hr with constant current at 360 mA with the cold pack and prechilled buffer. Membrane was immersed in blocking buffer (5% milk in 1×PBS) and blocked for 30 minutes. Antibodies and detection: Membranes incubated with primary antibodies at RT for 60 min with the following primary antibodies: IL-36× (R&D Systems), IL-36-gamma (Novus Biologicals), IL-4 (Thermo Fisher Scientific), IL-13 (Abcam), IL-31 (Thermo Fisher Scientific), 2 μg/mL blocking buffer, followed by washing in 0.05% Tween 20 in 1×PBS (3 times x 5 min). Membranes were incubated with secondary antibody (Abcam) for 60 min at room temperature, diluted in 2% milk in 1×PBS buffer for 60 min at room temp. Washed 3×5 min with 0.05% Tween 20 in 1×PBS. Signal detection with Western Blue stabilized substrate for alkaline phosphatase (Promega cat #S3841) following manufacturer recommended protocol. Western Blot sample analysis performed by GelAnalyzer 23.1.1 software. The Western Blot protein expression analysis for IL-4, IL-13, IL-31, IL-36 alpha, and IL-36 gamma are presented in FIGS. 2-6 and the Raw Integrated Density Data is provided in FIGS. 7-11. The IGA score for Groups I and II is provided in FIG. 12.

As shown in Table 21, the results of this study demonstrated that Formulation 1 exhibits broad spectrum cytokine inhibition. In particular, treatment with the composition of Formulation 1 resulted in a significant inhibition of several cytokines, including IL-4, IL-31, IL-36 alpha, and IL-36 gamma. Three days after pre-treatment, Formulation 1 achieved a 66.67% inhibition of IGA score (p value=0.0000000008). The study results also indicated that treatment with Formulation 1 resulted in a 67.7% inhibition of IL-31 (p value=0.0000011200), a 50.08% inhibition of IL-36 alpha (p value=3.08×10⁻¹³), a 49.05% inhibition of IL-36 gamma (p value=0.0000000435), and a 16.71% inhibition of IL-4 (p value=0.0000287). The results of this study also indicate that treatment pre-treatment with Formulation 1 reduces inflammation and disease severity in an eczema model by 66.67% in-vivo.

TABLE 21
IGA Score and Cytokine Inhibition after
Pretreatment with Formulation 1

| Variable | Percent Inhibition | P Value |
|---|---|---|
| IGA Score | 66.67% | 0.0000000008 |
| IL-31 | 67.7% | 0.00000112 |
| IL-36 alpha | 50.08% | 3.08 × 10⁻¹³ |
| IL-36 gamma | 49.05% | 0.0000000435 |
| IL-4 | 16.71% | 0.0000287 |
| IL-13 | 0.00% | 0.988 |

### Statements of the Disclosure:

Statement 1: A method of treating or preventing an inflammatory skin disease in a subject, the method comprising applying a petrolatum-based polyhexamethylene biguanide (PHMB) composition to the skin of a subject in need of treatment.
Statement 2: The method according to Statement 1, wherein the inflammatory skin disease is selected from the group consisting of eczema, psoriasis, rosacea, lichen planus, seborrheic dermatitis, acne vulgaris, urticaria, hidradenitis suppurativa, folliculitis, dermatitis herpetiformis, alopecia areata, and vitiligo.
Statement 3: The method according to Statement 1 or Statement 2, wherein the inflammatory skin disease is eczema.
Statement 4: A method of controlling, mitigating, or reducing inflammation in the skin of a subject, the method comprising applying a petrolatum-based polyhexamethylene biguanide (PHMB) composition to the skin of a subject in need of treatment.
Statement 5: A method of controlling, mitigating, or reducing the production of one or more pro-inflammatory cytokines in a subject, the method comprising applying a petrolatum-based polyhexamethylene biguanide (PHMB) composition to the skin of a subject in need of treatment.
Statement 6: The method according to Statement 5, wherein the one or more pro-inflammatory cytokines is selected from the group consisting of IL-36 alpha, IL-36 gamma, IL-31, and IL-4.
Statement 7: The method according to Statement 6, wherein the one or more pro-inflammatory cytokines is selected from the group consisting of IL-36 alpha, IL-36 gamma, and IL-31.
Statement 8: The method according to any one of Statements 1-7, wherein (1) the skin of the subject is colonized by a yeast, fungus, bacteria, or other microorganism; or (2) wherein the skin of the subject is determined to be colonized by a yeast, fungus, bacteria, or other microorganism; or (3) wherein a portion of the skin of the subject characterized as suffering from the inflammatory skin disease is colonized or determined to be colonized by a yeast, fungus, bacteria, or other microorganism.
Statement 9: The method according to any one of Statements 1-7, wherein the petrolatum-based PHMB composition prevents or treats inflammation in the skin of the subject by reducing the production or expression of one or more pro-inflammatory cytokines in the skin of the subject.
Statement 10: The method according to Statement 9, wherein the one or more pro-inflammatory cytokines is selected from the group consisting of IL-36 alpha, IL-36 gamma, IL-31, and IL-4.
Statement 11: The method according to Statement 8, wherein the yeast, fungus, bacteria, or other microorganism is selected from the group consisting of Staphylococcus aureus (S. aureus), Staphylococcus epidermis (S. epidermis), micrococci, diphtheroids (coryneforms), streptococci, gram-negative bacilli (such as Enterobacter, Klebsiella, Escherichia coli, Proteus spp., and Acinetobacter spp), Aspergillus, Penicillium, Cladosporium, Mucor, Propionibacterium acnes, P. granulosum, P. avidum, Corynebacterium spp., and Micrococcus luteus.
Statement 12: The method according to any one of Statements 8-10, wherein the yeast, fungus, bacteria, or other microorganism is Staphylococcus aureus (S. aureus).
Statement 13: The method according to any one of Statements 1-12, wherein the inflammation, inflammatory skin disease, or pro-inflammatory cytokines production is associated with or caused by Staphylococcus aureus (S. aureus).
Statement 14: The method according to any one of Statements 1-13, wherein the petrolatum-based PHMB composition comprises from about 0.1% to about 1% by weight PHMB.
Statement 15: The method according to any one of Statements 1-14, wherein the petrolatum-based PHMB composition comprises greater than about 80% by weight petrolatum.
Statement 16: The method according to any one of Statements 1-14, wherein the petrolatum-based PHMB composition comprises greater than about 85% by weight petrolatum.
Statement 17: The method according to any one of Statements 1-14, wherein the petrolatum-based PHMB composition comprises greater than about 90% by weight petrolatum.
Statement 18: The method according to any one of Statements 1-14, wherein the petrolatum-based PHMB composition comprises greater than about 95% by weight petrolatum.
Statement 19: The method according to any one of Statements 1-18, wherein the petrolatum-based PHMB composition excludes an added emulsifier.
Statement 20: The method according to any one of Statements 1-19, wherein the petrolatum-based PHMB composition further includes one or more cationic biocides selected from the group consisting of polyaminopropyl biguanide (PAPB), benzalkonium chloride, cetrimide, chlorhexidine, and any combination thereof.
Statement 21: The method according to Statement 20, wherein the total amount of the one or more cationic biocides is less than 1% by weight of the petrolatum-based PHMB composition.
Statement 22: The method according to any one of Statement 1-21, wherein the petrolatum-based PHMB composition is prepared by a process comprising: a) dissolving the PHMB in a polar solvent to give a PHMB solution; b) heating the petrolatum to a temperature sufficient to cause the petrolatum to melt to give a melted petrolatum and heating the PHMB solution to a temperature higher than the temperature of the melted petrolatum to give a heated PHMB solution; c) mixing the melted petrolatum and the heated PHMB solution to give a melted mixture; and d) cooling the melted mixture to give the petrolatum-based PHMB composition.
Statement 23: The method according to Statement 22, wherein the heated PHMB solution has a temperature that is about 1° C. to about 5° C. higher than the temperature of the melted petrolatum.
Statement 24: The method according to Statement 22 or Statement 23, wherein the PHMB is dissolved in a polar solvent to form a PHMB solution, the PHMB solution dispersed in the petrolatum to form a stable suspension.
Statement 25: The method according to any one of Statements 1-24, wherein the petrolatum-based PHMB composition further comprises at least one additional anti-fungal agent selected from the group consisting of terbinafine HCl, ciclopirox, ciclopirox olamine, fluconazole, itraconazole, ketoconazole, amorolfine, efinaconazole, clotrimazole, miconazole (miconazole nitrate) and any combination thereof.
Statement 26: The method according to Statement 25, wherein the petrolatum-based PHMB composition comprises from about 0.5% to about 1.5% by weight of the at least one additional anti-fungal agent.
Statement 27: The method according to any one of Statement 21-26, wherein the polar solvent is selected from the group consisting of water, ethanol, and any combination thereof.
Statement 28: A topical petrolatum-based composition for the treatment of an inflammatory skin disease in a subject, the composition comprising: from about 0.1% to about 1% by weight of cationic biocide, wherein the cationic biocide is selected from the group consisting of polyhexamethylene biguanide (PHMB), polyaminopropyl biguanide (PAPB), chlorhexidine (CHG), and any combination thereof; from about 2% to about 6% by weight polar solvent selected from the group consisting of water, ethanol, and any combination thereof; and greater than about 90% by weight petrolatum; wherein the cationic biocide is dissolved in the polar solvent and the polar solvent is suspended in the petrolatum without an added emulsifier.
Statement 29: The composition according to Statement 28, wherein the inflammatory skin disease is selected from the group consisting of eczema, psoriasis, rosacea, lichen planus, seborrheic dermatitis, acne vulgaris, urticaria, hidradenitis suppurativa, folliculitis, dermatitis herpetiformis, alopecia areata, and vitiligo.
Statement 30: The composition according to Statement 28 or Statement 29, wherein the inflammatory skin disease is eczema.
Statement 31: A topical petrolatum-based composition for controlling, mitigating, or reducing inflammation in the skin of a subject, the composition comprising: from about 0.1% to about 1% by weight of cationic biocide, wherein the cationic biocide is selected from the group consisting of polyhexamethylene biguanide (PHMB), polyaminopropyl biguanide (PAPB), chlorhexidine (CHG), and any combination thereof; from about 2% to about 6% by weight polar solvent selected from the group consisting of water, ethanol, and any combination thereof; and greater than about 90% by weight petrolatum; wherein the cationic biocide is dissolved in the polar solvent and the polar solvent is suspended in the petrolatum without an added emulsifier.
Statement 32: A petrolatum-based composition for controlling, mitigating, or reducing the production of one or more pro-inflammatory cytokines in the skin of a subject, the composition comprising: from about 0.1% to about 1% by weight of cationic biocide, wherein the cationic biocide is selected from the group consisting of polyhexamethylene biguanide (PHMB), polyaminopropyl biguanide (PAPB), chlorhexidine (CHG), and any combination thereof; from about 2% to about 6% by weight polar solvent selected from the group consisting of water, ethanol, and any combination thereof; and greater than about 90% by weight petrolatum; wherein the cationic biocide is dissolved in the polar solvent and the polar solvent is suspended in the petrolatum without an added emulsifier.
Statement 33: The composition according to Statement 32, wherein the one or more pro-inflammatory cytokines is selected from the group consisting of IL-36 alpha, IL-36 gamma, IL-31, and IL-4.
Statement 34: The composition according to Statement 33, wherein the one or more pro-inflammatory cytokines is selected from the group consisting of IL-36 alpha, IL-36 gamma, and IL-31.
Statement 35: The composition according to any one of Statements 28-34, wherein the skin of the subject is colonized by a yeast, fungus, bacteria, or other microorganism.
Statement 36: The composition according to any one of Statements 28-34, wherein the skin of the subject is determined to be colonized by a yeast, fungus, bacteria, or other microorganism.
Statement 37: The composition according to any one of Statements 28-34, wherein a portion of the skin of the subject characterized as suffering from the inflammatory skin disease is colonized or determined to be colonized by a yeast, fungus, bacteria, or other microorganism.
Statement 38: The composition according to any one of Statements 35-37, wherein the yeast, fungus, bacteria, or other microorganism is selected from the group consisting of Staphylococcus aureus (S. aureus), Staphylococcus epidermis (S. epidermis), micrococci, diphtheroids (coryneforms), streptococci, gram-negative bacilli (such as

Enterobacter, Klebsiella, Escherichia coli, Proteus spp., and Acinetobacter spp), Aspergillus, Penicillium, Cladosporium, Mucor, Propionibacterium acnes, P. granulosum, P. avidum, Corynebacterium spp., and Micrococcus luteus.
Statement 39: The composition according to any one of Statements 35-37, wherein the yeast, fungus, bacteria, or other microorganism is Staphylococcus aureus (S. aureus).
Statement 40: The composition according to any one of Statements 28-34, wherein the inflammation, inflammatory skin disease, or pro-inflammatory cytokines production is associated with or caused by Staphylococcus aureus (S. aureus).
Statement 41: A method of treating eczema in a subject, the method comprising topically applying a petrolatum-based polyhexamethylene biguanide (PHMB) composition to the skin of a subject in need of treatment.
Statement 42: A petrolatum-based composition for the topical treatment of eczema in a subject, the composition comprising: from about 0.1% to about 1% by weight of cationic biocide, wherein the cationic biocide is selected from the group consisting of polyhexamethylene biguanide (PHMB), polyaminopropyl biguanide (PAPB), chlorhexidine (CHG), and any combination thereof; from about 2% to about 6% by weight polar solvent selected from the group consisting of water, ethanol, and any combination thereof; and greater than about 90% by weight petrolatum; wherein the cationic biocide is dissolved in the polar solvent and the polar solvent is suspended in the petrolatum without an added emulsifier.
Statement 43: The composition according to Statement 42, wherein the composition treats eczema in the subject by reducing the production or expression of one or more pro-inflammatory cytokines in the skin of the subject.
Statement 44: The composition according to any one of Statements 28-43, wherein the petrolatum-based PHMB composition prevents or treats inflammation in the skin of the subject by reducing the production or expression of one or more pro-inflammatory cytokines in the skin of the subject.
Statement 45: The composition according to Statement 43 or Statement 44, wherein the one or more pro-inflammatory cytokines is selected from the group consisting of IL-36 alpha, IL-36 gamma, IL-31, and IL-4.
Statement 46: A method of treating or preventing a skin condition in a subject, the method comprising applying topically to the skin of the subject a petrolatum-based polyhexamethylene biguanide (PHMB) composition comprising PHMB and greater than about 80% by weight petrolatum; wherein the skin condition is selected from the group consisting of eczema, psoriasis, rosacea, seborrheic dermatitis, dermatitis herpetiformis, and acne.
Statement 47: The method according to Statement 46, wherein the PHMB is dissolved in a polar solvent to form a PHMB solution, the PHMB solution dispersed in the petrolatum to form a stable suspension.
Statement 48: The method according to Statement 46 or Statement 47, wherein the polar solvent is selected from the group consisting of water, ethanol, and any combination thereof.
Statement 49: The method according to any one of Statements 46-48, wherein the petrolatum-based PHMB composition comprises from about 0.1% to about 1% by weight PHMB.
Statement 50: The method according to any one of Statements 46-49, wherein the petrolatum-based PHMB composition comprises greater than about 85% by weight petrolatum.
Statement 51: The method according to any one of Statements 46-49, wherein the petrolatum-based PHMB composition comprises greater than about 90% by weight petrolatum.
Statement 52: The method according to any one of Statements 46-51, wherein the petrolatum-based PHMB composition excludes an added emulsifier.
Statement 53: The method according to any one of Statements 46-52, wherein the skin condition is eczema.
Statement 54: The method according to any one of Statements 46-52, wherein the skin condition is psoriasis.
Statement 55: The method according to any one of Statements 46-54, wherein a portion of the skin of the subject characterized as suffering from or determined to be colonized by a yeast, fungus, bacteria, or other microorganism.
Statement 56: The method according to Statement 55, wherein the yeast, fungus, bacteria, or other microorganism is Staphylococcus aureus (S. aureus).
Statement 57: The method according to any one of Statements 46-56, wherein the petrolatum-based PHMB composition is prepared by a process comprising: a) dissolving the PHMB in a polar solvent to give a PHMB solution; b) heating the petrolatum to a temperature sufficient to cause the petrolatum to melt to give a melted petrolatum and heating the PHMB solution to a temperature higher than the temperature of the melted petrolatum to give a heated PHMB solution; c) mixing the melted petrolatum and the heated PHMB solution to give a melted mixture; and d) cooling the melted mixture to give the petrolatum-based PHMB composition.
Statement 58: The method according to Statement 57, wherein the heated PHMB solution has a temperature that is about 1° C. to about 5° C. higher than the temperature of the melted petrolatum.
Statement 59: A method of treating or preventing red, irritated, or itchy skin in a subject, the method comprising applying topically to a portion of the skin of the subject in need of treatment a petrolatum-based polyhexamethylene biguanide (PHMB) composition comprising PHMB and greater than about 80% by weight petrolatum.
Statement 60: The method according to Statement 59, wherein the PHMB is dissolved in a polar solvent to form a PHMB solution, the PHMB solution dispersed in the petrolatum to form a stable suspension.
Statement 61: The method according to Statement 59 or Statement 60, wherein the petrolatum-based PHMB composition excludes an added emulsifier.
Statement 62: A method of treating or preventing a skin condition in a subject, the method comprising applying topically to the skin of the subject a petrolatum-based cationic biocide composition comprising: a cationic biocide selected from the group consisting of polyhexamethylene biguanide (PHMB), polyaminopropyl biguanide (PAPB), chlorhexidine (CHG), and any combination thereof; and greater than about 80% by weight petrolatum; wherein the skin condition is selected from the group consisting of eczema, psoriasis, rosacea, seborrheic dermatitis, dermatitis herpetiformis, and acne.
Statement 63: The method according to Statement 62, wherein the cationic biocide is dissolved in a polar solvent to form a cationic biocide solution, the cationic biocide solution dispersed in the petrolatum to form a stable suspension.
Statement 64: The method according to Statement 62 or Statement 63, wherein the petrolatum-based cationic biocide composition excludes an added emulsifier.
Statement 65: The method according to any one of Statements 62-64, wherein the petrolatum-based cationic biocide composition comprises: from about 0.1% to about 1% by weight of cationic biocide, wherein the cationic biocide is selected from the group consisting of polyhexamethylene biguanide (PHMB), polyaminopropyl biguanide (PAPB), chlorhexidine (CHG), and any combination thereof; from about 2% to about 6% by weight polar solvent selected from the group consisting of water, ethanol, and any combination thereof; and greater than about 90% by weight petrolatum; wherein the cationic biocide is dissolved in the polar solvent and the polar solvent is suspended in the petrolatum to form a stable composition.

What is claimed is:

1. A method of treating or preventing a skin condition in a subject, the method comprising applying topically to the skin of the subject a petrolatum-based polyhexamethylene biguanide (PHMB) composition comprising PHMB and greater than about 80% by weight petrolatum; wherein the skin condition is selected from the group consisting of eczema, psoriasis, rosacea, seborrheic dermatitis, dermatitis herpetiformis, and acne.

2. The method according to claim 1, wherein the PHMB is dissolved in a polar solvent to form a PHMB solution, the PHMB solution dispersed in the petrolatum to form a stable suspension.

3. The method according to claim 2, wherein the polar solvent is selected from the group consisting of water, ethanol, and any combination thereof.

4. The method according to claim 1, wherein the petrolatum-based PHMB composition comprises from about 0.1% to about 1% by weight PHMB.

5. The method according to claim 1, wherein the petrolatum-based PHMB composition comprises greater than about 85% by weight petrolatum.

6. The method according to claim 1, wherein the petrolatum-based PHMB composition comprises greater than about 90% by weight petrolatum.

7. The method according to claim 1, wherein the petrolatum-based PHMB composition excludes an added emulsifier.

8. The method according to claim 1, wherein the skin condition is eczema.

9. The method according to claim 1, wherein the skin condition is psoriasis.

10. The method according to claim 1, wherein a portion of the skin of the subject characterized as suffering from or determined to be colonized by a yeast, fungus, bacteria, or other microorganism.

11. The method according to claim 10, wherein the yeast, fungus, bacteria, or other microorganism is Staphylococcus aureus (S. aureus).

12. The method according to claim 1, wherein the petrolatum-based PHMB composition is prepared by a process comprising:
a) dissolving the PHMB in a polar solvent to give a PHMB solution;
b) heating the petrolatum to a temperature sufficient to cause the petrolatum to melt to give a melted petrolatum and heating the PHMB solution to a temperature higher than the temperature of the melted petrolatum to give a heated PHMB solution;
c) mixing the melted petrolatum and the heated PHMB solution to give a melted mixture; and
d) cooling the melted mixture to give the petrolatum-based PHMB composition.

13. The method according to claim 12, wherein the heated PHMB solution has a temperature that is about 1° C. to about 5° C. higher than the temperature of the melted petrolatum.

14. A method of treating or preventing red, irritated, or itchy skin in a subject, the method comprising applying topically to a portion of the skin of the subject in need of treatment a petrolatum-based polyhexamethylene biguanide (PHMB) composition comprising PHMB and greater than about 80% by weight petrolatum.

15. The method according to claim 14, wherein the PHMB is dissolved in a polar solvent to form a PHMB solution, the PHMB solution dispersed in the petrolatum to form a stable suspension.

16. The method according to claim 15, wherein the petrolatum-based PHMB composition excludes an added emulsifier.

17. A method of treating or preventing a skin condition in a subject, the method comprising applying topically to the skin of the subject a petrolatum-based cationic biocide composition comprising:
a cationic biocide selected from the group consisting of polyhexamethylene biguanide (PHMB), polyaminopropyl biguanide (PAPB), chlorhexidine (CHG), and any combination thereof; and
greater than about 80% by weight petrolatum;
wherein the skin condition is selected from the group consisting of eczema, psoriasis, rosacea, seborrheic dermatitis, dermatitis herpetiformis, and acne.

18. The method according to claim 17, wherein the cationic biocide is dissolved in a polar solvent to form a cationic biocide solution, the cationic biocide solution dispersed in the petrolatum to form a stable suspension.

19. The method according to claim 18, wherein the petrolatum-based cationic biocide composition excludes an added emulsifier.

20. The method according to claim 18, wherein the petrolatum-based cationic biocide composition comprises:
from about 0.1% to about 1% by weight of cationic biocide, wherein the cationic biocide is selected from the group consisting of polyhexamethylene biguanide (PHMB), polyaminopropyl biguanide (PAPB), chlorhexidine (CHG), and any combination thereof;
from about 2% to about 6% by weight polar solvent selected from the group consisting of water, ethanol, and any combination thereof; and
greater than about 90% by weight petrolatum;
wherein the cationic biocide is dissolved in the polar solvent and the polar solvent is suspended in the petrolatum to form a stable composition.
